(19) United States
(12) Patent Application Publication
(10) Pub. No.: US 2024/0335394 A1
(43) Pub. Date: Oct. 10, 2024

(54) Use Of Methanol And Prodrugs Thereof In Therapy

(71) Applicant: Ludwig Institute for Cancer Research Ltd, Zurich (CH)

(72) Inventors: Kellen Olszewski, Philadelphia, PA (US); Joshua D. Rabinowitz, Princeton, NJ (US); Hahn Kim, Princeton, NJ (US)

(21) Appl. No.: 18/579,221

(22) PCT Filed: Jul. 15, 2022
(86) PCT No.: PCT/US2022/073769

§ 371 Date: Jan. 12, 2024

Related U.S. Application Data
(60) Provisional application No. 63/265,853, filed on Dec. 22, 2021.
(60) Provisional application No. 63/222,156, filed on Jul. 15, 2021.

(51) Int. Cl. A61K 31/045; A61K 9/00; A61K 45/06; A61P 35/00; C07K 16/28

(52) U.S. Cl. CPC A61K 31/045; A61K 9/0053; A61K 45/06; A61P 35/00; C07K 16/2818; C07K 16/2827

(57) ABSTRACT

Provided herein are methods of using methanol, or a prodrug thereof, in therapy, e.g., for promoting an immune response, treating cancer, enhancing an immunotherapy and/or treating immune dysfunction. Also provided herein are methanol prodrugs of the following structural formula: Formula (I), or a pharmaceutically acceptable salt thereof, wherein values for the variables (e.g., R, X, R2, n) are as described herein.

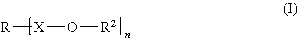

### RELATED APPLICATIONS

This application claims the benefit of U.S. Provisional Application No. 63/222,156, filed on Jul. 15, 2021, and U.S. Provisional Application No. 63/265,853, filed on Dec. 22, 2021. The entire teachings of these applications are incorporated herein by reference.

### BACKGROUND

Tumor-resident and aged T-cells are 1C-starved, which compromises their function. Formate supplies one-carbon (1C) units directly to 1C metabolism. Newman and Maddocks, Br J Canc 116, pages 1499-1504 (2017). Formate supplementation rescues T-cell activity, but formate is too rapidly eliminated to dose directly. See Hanzlik, R. P., et al., Drug Metab. Dispos. 33(2):282-286 (2005). In addition, formate is known to be the toxic metabolite of methanol, causing formic acidosis and lactic acidosis upon prolonged exposure, particularly in primates, which have low liver formate metabolism. Clay, K. L., et al., Tox Appl Pharmacol 34(1):49-61 (1975).

Accordingly, there is a need for therapies that provide an extended increase in formate levels and have an acceptable toxicity profile.

### SUMMARY

Provided herein are compounds of Structural Formula I:

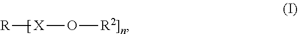

or a pharmaceutically acceptable salt thereof, wherein values for the variables (e.g., R, X, R², n) are as described herein.

Also provided herein are compounds of Structural Formula II:

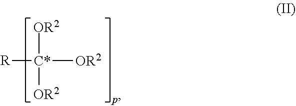

or a pharmaceutically acceptable salt thereof, wherein values for the variables (e.g., R, R², p) are as described herein.

Also provided herein are pharmaceutical compositions comprising a compound of the disclosure (e.g., a compound of Structural Formula I or II, or a pharmaceutically acceptable salt thereof) and a pharmaceutically acceptable carrier.

Also provided herein are pharmaceutical combinations comprising a compound of the disclosure (e.g., a compound of Structural Formula I or II, or a pharmaceutically acceptable salt thereof) and an additional therapeutic agent.

Also provided herein is a method of promoting the survival, proliferation or activation of an immune cell, comprising contacting the immune cell with an effective amount of methanol, or a prodrug thereof (e.g., a compound of Structural Formula I or II, or a pharmaceutically acceptable salt thereof).

Also provided herein are methods of promoting an immune response; treating cancer; enhancing an immunotherapy; and/or treating an immune dysfunction in a subject in need thereof, comprising administering to the subject a therapeutically effective amount of methanol, or a prodrug thereof (e.g., a compound of Structural Formula I or II, or a pharmaceutically acceptable salt thereof).

Also provided herein is a compound of the disclosure (e.g., a compound of Structural Formula I or II, or a pharmaceutically acceptable salt thereof) for use in promoting an immune response; treating cancer; enhancing an immunotherapy; and/or treating an immune dysfunction. Also provided herein is a composition (e.g., pharmaceutical composition) for use in promoting an immune response; treating cancer; enhancing an immunotherapy; and/or treating an immune dysfunction, wherein the composition comprises a compound of the disclosure (e.g., a compound of Structural Formula I or II, or a pharmaceutically acceptable salt thereof). Also provided herein is a use of a compound of the disclosure (e.g., a compound of Structural Formula I or II, or a pharmaceutically acceptable salt thereof) for the manufacture of a medicament for promoting an immune response; treating cancer; enhancing an immunotherapy; and/or treating an immune dysfunction.

It is shown herein that methanol can provide extended release of formate and synergize with immunotherapy in a mouse model of cancer at levels expected to be at least 20-fold lower than those observed to produce symptomatic toxicity in primates.

### BRIEF DESCRIPTION OF THE DRAWINGS

The foregoing will be apparent from the following more particular description of example embodiments.

FIG. 1 shows liver methanol metabolism.

FIG. 2A shows tumor volume in mice bearing syngeneic MC38 tumors and dosed with anti-PD1 antibody alone or in combination with sodium formate according to Example 1.

FIG. 2B shows tumor volume in mice bearing syngeneic MC38 tumors and dosed with anti-PD1 antibody alone or in combination with methanol according to Example 1.

FIG. 2C shows survival of mice bearing syngeneic MC38 tumors and dosed with anti-PD1 antibody alone, or anti-PD1 antibody in combination with sodium formate or methanol according to Example 1.

FIG. 3 shows average tumor volumes of mice in the MC38 rechallenge described in Example 2.

FIG. 4A is a waterfall plot, and shows tumor volume in mice bearing syngeneic MC38 tumors and dosed with anti-PD-1 antibody alone or in combination with HOCH₃ (methanol) or methanol-d₃ according to Example 4 (volume increases truncated at 500% for display purposes).

FIG. 4B shows plasma ¹³C-formate levels in C57BL/6 mice dosed with 2 mg/kg PO HOCH₃ (methanol) or methanol-d₄.

FIG. 4C shows expected liver metabolism of methanol-d₄.

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

When introducing elements disclosed herein, the articles “a,” “an,” “the,” and “said” are intended to mean that there are one or more of the elements. Further, the one or more elements may be the same or different.

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., +10%, +5% or +1% of a given value. It is to be understood that the term “about” can precede any particular value specified herein, except for particular values used in the Exemplification.

“Aliphatic” refers to a non-aromatic, branched- or straight-chain and/or cyclic, hydrocarbon radical having the specified number of carbon atoms. Thus, “(C₁-C₁₅)aliphatic” refers to an aliphatic radical having from 1-15 carbon atoms. In some embodiments, aliphatic is (C₁-C₂₅)aliphatic, e.g., (C₁-C₂₀)aliphatic, (C₁-C₁₅)aliphatic, (C₁-C₁₀)aliphatic, (C₁-C₅)aliphatic or (C₁-C₃)aliphatic. “Aliphatic” can be saturated, or contain one or more units of unsaturation (e.g., carbon-carbon double bonds). Examples of aliphatic include alkyl, alkenyl, alkynyl and cycloalkyl. In some embodiments, aliphatic is saturated aliphatic, for example, (C₁-C₂₅)saturated aliphatic, (C₁-C₁₅)saturated aliphatic, (C₁-C₁₀)saturated aliphatic or (C₁-C₅)saturated aliphatic. In some embodiments, aliphatic is alkyl or alkenyl. In some embodiments, aliphatic is alkyl. In some embodiments, aliphatic is optionally substituted, e.g., with one or more substituents described herein.

“Alkyl” refers to a saturated, branched- or straight-chain, aliphatic radical having the specified number of carbon atoms. Thus, “(C₁-C₁₅)alkyl” refers to a radical having from 1-15 carbon atoms in a branched or linear arrangement. In some embodiments, alkyl is (C₁-C₂₅)alkyl, e.g., (C₁-C₂₀)alkyl, (C₁-C₁₅)alkyl, (C₁-C₁₀)alkyl, (C₁-C₅)alkyl or (C₁-C₃)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some embodiments, alkyl is optionally substituted, e.g., with one or more substituents described herein.

“Alkylether” refers to a saturated, branched- or straight-chain and/or cyclic, aliphatic radical having the specified number of atoms in its chain and/or cycle, wherein one or more carbon atoms has each been replaced with an oxygen atom and wherein each oxygen atom in the chain and/or cycle is bound to two carbon atoms. Thus, “(C₁-C₁₅)alkylether” refers to a radical having from 1-15 carbon and oxygen atoms in a branched or linear and/or cyclic arrangement wherein each oxygen atom in the chain and/or cycle is bound to two carbon atoms. In some embodiments, alkylether is (C₁-C₂₅)alkylether, e.g., (C₁-C₂₀)alkylether, (C₁-C₁₅)alkylether, (C₁-C₁₀)alkylether, (C₁-C₅)alkylether or (C₁-C₃)alkylether. Examples of alkylether groups include tetrahydrofuran, tetrahydropyran, 2-methyl-3-((6-methyltetrahydro-2H-pyran-2-yl)oxy)tetrahydro-2H-pyran, and the like. In some embodiments, alkylether is optionally substituted, e.g., with one or more substituents described herein.

“Alkenyl” refers to a branched- or straight-chain, aliphatic radical having the specified number of carbon atoms and at least one (e.g., one, two, three, four, five, etc.) carbon-carbon double bonds. Thus, “(C₁-C₁₅)alkenyl” refers to a radical having from 1-15 carbon atoms and at least one carbon-carbon double bond in a branched or linear arrangement. In some embodiments, alkenyl is (C₁-C₂₅)alkenyl, e.g., (C₁-C₂₀)alkenyl, (C₁-C₁₅)alkenyl, (C₁-C₁₀)alkenyl, (C₁-C₅)alkenyl or (C₁-C₃)alkenyl. Examples of alkyl groups include vinyl, allyl, and the like. In some embodiments, alkenyl is optionally substituted, e.g., with one or more substituents described herein.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Aryl” refers to a monocyclic or polycyclic (e.g., bicyclic, tricyclic), carbocyclic, aromatic ring system having the specified number of ring atoms, and includes aromatic ring(s) fused to non-aromatic rings, as long as one of the fused rings is an aromatic hydrocarbon. Thus, “(C₆-C₁₅)aryl” means an aromatic ring system having from 6-15 ring atoms. In some embodiments, aryl is (C₆-C₂₅)aryl, for example, (C₆-C₂₀)aryl, (C₆-C₁₅)aryl, (C₆-C₁₂)aryl or (C₆-C₁₀)aryl. Examples of aryl include phenyl, naphthyl and fluorenyl. In some embodiments, aryl is phenyl or fluorenyl. In some embodiments, aryl is optionally substituted, e.g., with one or more substituents described herein.

“Deuteromethanol” or “deuterated methanol” refers to a deuterium isotopologue of methanol, as that term is used herein. Examples of deuteromethanol include HOCD₃, HOCH₂D, HOCHD₂, HO¹³CD₃, HO¹³CH₂D and HO¹³CHD₂. Further examples of deuteromethanol include DOCD₃, DOCH₂D, DOCHD₂, DO¹³CH₃, DO¹³CD₃, DO¹³CH₂D and DO¹³CHD₂. In some embodiments, deuteromethanol is HOCD₃, DOCD₃, HO¹³CD₃ or DO¹³CD₃. In some embodiments, deuteromethanol is HOCD₃ or HO¹³CD₃. In some embodiments, deuteromethanol is HOCD₃ or DOCD₃. In some embodiments, deuteromethanol is HOCD₃. In some embodiments, deuteromethanol is DOCD₃.

“Halogen” and “halo” are used interchangeably herein and each refers to fluorine, chlorine, bromine, or iodine. In some embodiments, halo is fluoro, chloro or bromo. In some embodiments, halo is fluoro.

“Hetero” refers to an atom that is not carbon or hydrogen. Examples of heteroatoms include, but are not limited to, nitrogen, oxygen, sulfur, boron, silicon, and the like. In some embodiments, hetero is selected from nitrogen, oxygen or sulfur. In some embodiments, hetero is selected from nitrogen or oxygen.

“Heteroaliphatic,” as used herein, refers to a non-aromatic, branched- or straight-chain and/or cyclic, hydrocarbon radical having the specified number of carbon atoms, wherein at least one carbon atom has been replaced with a heteroatom (e.g., N, S and/or O; N or O). Thus, “(C₁-C₁₀)heteroaliphatic” refers to a heteroaliphatic radical having from one to 10 atoms. In some embodiments, heteroaliphatic is (C₁-C₂₅)heteroaliphatic, for example, (C₁-C₁₅)heteroaliphatic, (C₁-C₁₀)heteroaliphatic, (C₁-C₆)heteroaliphatic, (C₁-C₅)heteroaliphatic or (C₁-C₃)heteroaliphatic. “Heteroaliphatic” can be saturated or contain one or more units of unsaturation. Examples of heteroaliphatic include heteroalkyl (e.g., alkylether) and heterocyclyl. In some embodiments, heteroaliphatic is heteroalkyl. In some embodiments, heteroaliphatic is optionally substituted, e.g., with one or more substituents described herein.

“Hydroxy” refers to —OH or —OD. In some embodiments, hydroxy is —OH.

“Formaldehyde” refers to HC(O)H, or an isotopologue thereof, for example, a deuterium isotopologue and/or ¹³C isotopologue thereof. Examples of isotopologues of HC(O)H include HC(O)D, DC(O)D, H¹³C(O)H, D¹³C(O)H and D¹³C(O)D.

“Formyl” refers to —C(O)H, or an isotopologue thereof, for example, a deuterium isotopologue and/or ¹³C isotopologue thereof. Examples of isotopologues of —C(O)H include —C(O)D, —¹³C(O)H and —¹³C(O)D. In some embodiments, formyl is —C(O)H, —C(O)D, —¹³C(O)H or —¹³C(O)D. In some embodiments, formyl is —C(O)H or —C(O)D. In some embodiments, formyl is —C(O)H or —¹³C(O)H.

“Hydrolyzable formyl” refers to formyl, as that term is described herein, covalently attached to a nitrogen or oxygen atom. Examples of hydrolyzable formyl include —O—C(O)H and —N(C(O)H)₂, or an isotopologue of the foregoing.

“Methanol” refers to HOCH₃, or an isotopologue thereof, for example, a deuterium isotopologue and/or ¹³C isotopologue thereof. Examples of isotopologues of HOCH₃ include HOCD₃, HOCH₂D, HOCHD₂, HO¹³CH₃, HO¹³CD₃, HO¹³CH₂D and HO¹³CHD₂. Further examples of isotopologues of HOCH₃ include DOCD₃, DOCH₂D, DOCHD₂, DO¹³CH₃, DO¹³CD₃, DO¹³CH₂D and DO¹³CHD₂. In some embodiments, methanol is HOCH₃, HOCD₃, DOCD₃, HO¹³CH₃, HO¹³CD₃ or DO¹³CD₃. In some embodiments, methanol is HOCH₃, HOCD₃, HO¹³CH₃ or HO¹³CD₃. In some embodiments, methanol is HOCH₃, HOCD₃ or DOCD₃. In some embodiments, methanol is HOCH₃ or HOCD₃. In some embodiments, methanol is HOCH₃. In some embodiments, methanol is HOCD₃. In some embodiments, methanol is DOCD₃.

“Methoxide” refers to H₃CO—, or an isotopologue thereof, for example, a deuterium isotopologue and/or ¹³C isotopologue thereof. Examples of isotopologues of H₃CO⁻ include OCD₃, —OCH₂D, —OCHD₂, —O¹³CH₃, —O¹³CD₃, —O¹³CH₂D and —O¹³CHD₂. In some embodiments, methoxide is —OCH₃, —OCD₃, —O¹³CH₃ or —O¹³CD₃. In some embodiments, methoxide is —OCH₃ or —OCD₃. In some embodiments, methoxide is —OCH₃. In some embodiments, methoxide is —OCD₃.

“Methyl ortho ester” refers to —C(OCH₃)₃, or an isotopologue thereof, for example, a deuterium and/or ¹³C isotopologue thereof. Examples of isotopologues of —C(OCH₃)₃ include —C(OCD₃)₃, —C(O¹³CH₃)₃ and —C(O¹³CD₃)₃.

“Geminal dimethoxy” refers to replacement of two hydrogen atoms bound to a single carbon atom with two methoxides, as that term is used herein.

“Methyl ester” refers to —C(O)OCH₃, or an isotopologue thereof, for example, a deuterium and/or ¹³C isotopologue thereof. Examples of isotopologues of —C(O)OCH₃ include —C(O)OCD₃, —C(O)O¹³CH₃ and —C(O)O¹³CD₃.

The term “substituted” refers to replacement of a hydrogen atom with a suitable substituent. Typically, the suitable substituent replaces a hydrogen atom bound to a carbon atom, but a substituent may also replace a hydrogen bound to a heteroatom, such as a nitrogen atom. When more than two or more hydrogen atoms are each replaced with an independently selected substituent, the substituents can be the same or different. It will be understood that “substitution” or “substituted with” includes the implicit proviso that such substitution is in accordance with permitted valence of the substituted atom. It is also preferred that the substituent, and the substitution, result in a stable compound, e.g., which does not spontaneously undergo transformation such as by rearrangement, cyclization, elimination, etc.

The term “optionally substituted”, as used herein, means that substitution is optional and, therefore, it is possible for the atom or moiety designated as “optionally substituted” to be unsubstituted or substituted. An “optionally substituted” group is, in some embodiments, substituted with 0-5 (e.g., 0-3, 0, 1, 2, 3, 4, 5) substituents. Unless otherwise indicated, e.g., as with the terms “substituted” or “optionally substituted,” a group designated herein is unsubstituted.

As used herein, the term “compound of the disclosure” refers to a prodrug described herein (e.g., methanol, methoxide, formaldehyde, a prodrug of methanol, such as a compound of any structural formula depicted herein (e.g., a compound of structural formula I or a subformula thereof)), as well as isomers, such as stereoisomers (including diastereoisomers, enantiomers and racemates) and tautomers thereof, isotopologues thereof, and inherently formed moieties (e.g., polymorphs and/or solvates, such as hydrates) thereof. When a moiety is present that is capable of forming a salt, then salts are included as well, in particular, pharmaceutically acceptable salts.

Compounds of the disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers (including cis and trans double bond isomers), conformational isomers (including rotamers and atropisomers), tautomers) and intermediate mixtures, with all possible isomers and mixtures thereof being included, unless otherwise indicated.

When a disclosed compound is depicted by structure without indicating the stereochemistry, and the compound has one or more chiral centers, it is to be understood that the structure encompasses one enantiomer or diastereomer of the compound separated or substantially separated from the corresponding optical isomer(s), a racemic mixture of the compound, and mixtures enriched in one enantiomer or diastereomer relative to its corresponding optical isomer(s). When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” can be used to indicate the absolute configuration of substituents around one or more chiral carbon atoms. D- and L- can also be used to designate stereochemistry.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 ... )}{D 1 + ( D 2 + D 3 + D 4 ... )} \vert \times 100 \% ,$$

where D1 and (D2+D3+D4 . . . ) represent the respective fractions of each diastereomer in a mixture, such that D1+(D2+D3+D4 . . . )=1. A diastereomer may be present in a de of at least or about 50%, about 60%, about 70%, about 80%, about 90%, about 95%, about 98%, about 99% or about 99.9%.

The term “isotopologue” refers to a molecule that differs from a reference molecule only in its isotopic composition.

Certain atoms naturally occur in various isotopic forms. Natural isotopic abundance describes the relative abundance of the various naturally-occurring isotopes of a given atom. Thus, it will be understood that a population of molecules represented by a particular chemical structure will typically contain isotopologues of the particular chemical structure. The relative amount of such isotopologues will depend upon a number of factors, such as relative natural isotopic abundance, the isotopic purity of reagents used to make the compound and the efficiency of incorporation of isotopic atoms in the various synthetic steps used to prepare the compound. In certain embodiments, the amount of such isotopologues in toto will be less than 49.9%, for example, less than 47.5%, less than 40%, less than 32.5%, less than 25%, less than 17.5%, less than 10%, less than 5%, less than 3%, less than 1%, or less than 0.5%.

In the compounds of the disclosure, any atom not specifically designated as a particular isotope is meant to represent any stable isotope of that atom. In preferred embodiments, any atom not specifically designated as a particular isotope is present at its natural isotopic abundance, which can conveniently be denoted by the symbol for the element, e.g., C for carbon, H for hydrogen.

Unless otherwise stated, when a position is designated specifically as “H” or “¹H”, the position is understood to have hydrogen at its natural isotopic abundance. However, in certain embodiments, a position designated specifically as “H” or “¹H” has at least 80%, at least 90%, at least 95%, at least 96%, at least 97%, at least 98%, or at least 99% ¹H. In some embodiments, a position designated specifically as “H” or “¹H” has ≤20% deuterium, ≤10% deuterium, ≤5% deuterium, ≤4% deuterium, ≤3% deuterium, ≤2% deuterium, or ≤1% deuterium.

Unless otherwise stated, when a position is designated specifically as “D” or “2H” or “deuterium”, the position is understood to have deuterium at an abundance that is at least 3,340 times greater than the natural abundance of deuterium, which is 0.015%, such that the position has at least 50.1% deuterium. In some embodiments, a position designated specifically as “D” or “²H” or “deuterium” has at least 55%, at least 65%, at least 75%, at least 80%, at least 85%, at least 90%, at least 95%, at least 96%, at least 97%, at least 98%, at least 99% or at least 99.5% deuterium. In some embodiments, a position designated specifically as “D” or “²H” or “deuterium” has ≤20% ¹H, ≤10% ¹H, ≤5% ¹H, ≤4% ¹H, ≤3% ¹H, ≤2% ¹H, or ≤1% ¹H.

Unless otherwise stated, when a position is designated specifically as “C” or “¹²C”, the position is understood to have carbon at its natural isotopic abundance. However, in certain embodiments, a position designated specifically as “C” or “¹²C” has at least 80%, at least 90%, at least 95%, at least 96%, at least 97%, at least 98%, or at least 99% ¹²C. In some embodiments, a position designated specifically as “C” or “¹²C” has ≤20% ¹³C, ≤10% ¹³C, ≤5% ¹³C, ≤4% ¹³C, ≤3% ¹³C, ≤2% ¹³C, or ≤1% ¹³C.

Unless otherwise stated, when a position is designated specifically as “¹³C”, the position is understood to have ¹³C at an abundance that is at least 45.5 times greater than the natural abundance of ¹³C, which is 1.1%, such that the position has at least 50.1% ¹³C. In some embodiments, a position designated specifically as “¹³C” has at least 55%, at least 65%, at least 75%, at least 80%, at least 85%, at least 90%, at least 95%, at least 96%, at least 97%, at least 98%, at least 99% or at least 99.5% ¹³C. In some embodiments, a position designated specifically as “¹³C” has ≤20% ¹²C, ≤10% ¹²C, ≤5% ¹²C, ≤4% ¹²C, ≤3% ¹²C, ≤2% ¹²C, or ≤1% ¹²C. It is expected that ¹³C-labeled compounds described herein will aid in monitoring blood methanol/formate levels, e.g., during therapeutic use of the compounds.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include pharmaceutically acceptable acid addition salts and pharmaceutically acceptable base addition salts.

Examples of pharmaceutically acceptable acid addition salts are salts of an amino group formed with inorganic acids such as hydrochloric acid, hydrobromic acid, phosphoric acid, sulfuric acid and perchloric acid, or with organic acids, such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like. Either the mono-, di- or tri-acid salts can be formed, and such salts can exist in either a hydrated, solvated or substantially anhydrous form.

Pharmaceutically acceptable base addition salts include salts formed with inorganic bases, such as alkali metal, alkaline earth metal, and ammonium bases, and salts formed with aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable base addition salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating,” as used herein, refers to taking steps to deliver a therapy to a subject, such as a mammal, in need thereof (e.g., as by administering to a subject one or more therapeutic agents). For example, a subject may be treated with methanol and/or methoxide in accordance with the present disclosure by administering to the subject methanol or a prodrug of methanol. “Treating” includes inhibiting a disease or condition (e.g., as by slowing or stopping its progression or causing regression of the disease or condition), and relieving the symptoms resulting from a disease or condition.

An “effective amount” is an amount effective, at dosages and for periods of time necessary, to achieve a desired result (e.g., a desired therapeutic result, a desired in vitro result). Methanol is often used in cellular fixation and/or permeabilization methods. In fixation methods, methanol is thought to act by dehydrating cells and precipitating proteins. Methanol is thought to permeabilize cells by dissolving lipids in the cell membrane. Preferably, an “effective amount” does not result in fixation and/or permeabilization of cells contacted with the amount, at dosages and for periods of time necessary, to achieve the desired result.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual. A therapeutically effective amount of an agent to be administered can be determined by a clinician of ordinary skill using the guidance provided herein and other methods known in the art.

It is well-documented that formate is the toxic metabolite of methanol, causing formic acidosis and, due to its weakly inhibitory effect on cytochrome c oxidase, lactic acidosis. Liver metabolism is particularly slow in primates, rendering them uniquely sensitive to formate toxicity. In humans, a lethal dose of methanol is thought to be in the range of 0.3-1.0 g/kg, and in primates, symptomatic toxicity is observed at formate levels greater than 8 mM. Clay, K. L., et al., Tox Appl Pharmacol 34(1):49-61 (1975). Preferably, a “therapeutically effective amount” does not cause undue toxicity (e.g., symptomatic toxicity) and/or does not produce side effects that are not commensurate with a reasonable benefit/risk ratio.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human, such as an aged human (e.g., a human aged greater than: 50, 55, 60, 65, 70, 75, 80, 85, or 90 years-old).

### Prodrugs and Methanol Prodrugs

Provided herein are prodrugs (e.g., methanol prodrugs), or a pharmaceutically acceptable salt thereof.

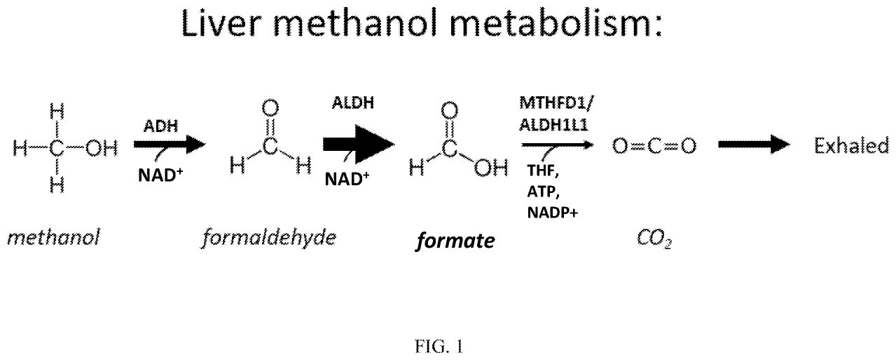

The term “prodrug,” used herein, refers to a molecule that can be hydrolyzed, oxidized, metabolized and/or otherwise transformed under suitable (e.g., biological) conditions to provide at least one one-carbon (1C) unit suitable for use in 1C metabolism in a subject per molecule. For example, without wishing to be bound by any particular theory, it is believed that a methyl ester may be de-esterified by a methyl esterase to provide methanol. It will be appreciated that, due to methanol liver metabolism (see FIG. 1), methanol itself is a prodrug, as that term is used herein, as is formaldehyde. “Prodrug” is meant to encompass both such molecules, as well as prodrugs that provide methanol or methoxide (“methanol prodrugs” or “prodrug of methanol”) and/or formaldehyde or formyl.

Prodrugs may become active upon transformation, or they may have activity in their untransformed form. A prodrug may undergo reduced metabolism under physiological conditions (e.g., due to the presence of a hydrolyzable group), for example, thereby resulting in improved circulating half-life of the prodrug (e.g., in the blood) and/or improved duration of action, and/or a prodrug may have other advantageous properties, such as improved water solubility and/or improved onset of action. Prodrugs can be absorbed, for example, through passive diffusion (as when a prodrug has a high degree of formylation), or through active transport, such as Na⁺/glucose transport (as when a prodrug has a relatively low degree of formylation). Prodrugs can be prepared using well-known methods, such as those described in Burger's Medicinal Chemistry and Drug Discovery (1995) 172-178, 949-982 (Manfred E. Wolff ed., 5^{th} ed).

Prodrugs can be derived from metabolites, amino acid, polyols, food additives and/or Generally Recognized As Safe (GRAS) substances such that, for example, the prodrug releases the metabolite, amino acid, polyol, food additive and/or GRAS substance upon transformation. It is expected that the metabolite, amino acid, polyol, food additive and/or GRAS substance thus released will be benign to the subject. In addition, functional groups inherent in these compounds, including hydroxy, amino and carboxy functional groups, may serve as convenient attachment points for creation of moieties capable of being transformed into 1C units suitable for use in 1C metabolism. Thus, in some embodiments, a prodrug (e.g., methanol prodrug) comprises a residue (e.g., a residue of a metabolite, amino acid, polyol, food additive and/or GRAS substance).

“Residue,” used herein with respect to a prodrug, refers to that portion of a referenced compound (minus those portion(s) of the referenced compound which form part of a functional group that is transformed under suitable conditions to provide a 1C unit suitable for use in 1C metabolism, such as moiety [X—O—R²]ₙ in Structural Formula I, a methyl ortho ester, a geminal dimethoxy) present in a prodrug that releases the referenced compound upon transformation. For example, the following methanol prodrug:

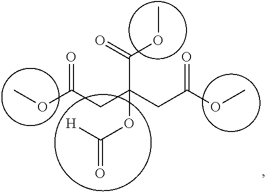

comprises a residue of citric acid because, upon hydrolysis of the three methyl esters and the formyl ester, it will release citric acid. Examples of residues include residues of metabolites, amino acid, polyols, food additives and/or GRAS substances. In some embodiments, a residue is a residue of a metabolite, amino acid, polyol, food additive or GRAS substance. Examples of metabolites include citric acid, isocitric acid, aconitic acid, alpha-ketoglutaric acid, fumaric acid, malic acid, succinic acid, lactic acid and pyruvic acid. Examples of amino acids include glycine, alanine, lysine, aspartic acid, glutamic acid, serine, threonine, asparagine and glutamine. Examples of polyols include glycerol, erythritol, xylitol, sorbitol, ribose, 2-deoxyribose, fructose, glucose, galactose, mannose, allose, altrose, gulose, idose, talose, xylose, maltitol, isomalt and sucrose. Examples of food additives include tartaric acid, ascorbic acid and pectin. Examples of GRAS substances include acetic acid, adipic acid, oleic acid, palmitic acid, pantothenic acid, stearic acid, octanoic acid, cholic acid, deoxycholic acid, glycocholic acid, linoleic acid, propionic acid, taurocholic acid, sorbic acid, pectin, pectinic acid, ascorbic acid, benzoic acid, gluconic acid and alginic acid. In some embodiments, a residue is a residue of a carboxylic acid, such as an endogenous carboxylic acid. Examples of endogenous carboxylic acids include formic acid, pyruvic acid, oxoglutaric acid (e.g., 2-oxoglutaric acid), oxalosuccinic acid, malonic acid, citric acid, 1-hydroxypropane-1,2,3-tricarboxylic acid, succinic acid, 2-oxosuccinic acid, glycine, glutamic acid, aspartic acid and alanine.

Prodrugs can also or alternatively be derived from endogenous and/or naturally-occurring, or synthetic compounds, such as endogenous carboxylic acids.

Transformation (e.g., hydrolysis) of a prodrug into a 1C unit suitable for use in 1C metabolism may be achieved enzymatically, chemically and/or spontaneously. Functional groups particularly suitable for transformation (e.g., hydrolysis) under suitable (e.g., biological) conditions to provide a 1C unit suitable for use in 1C metabolism include functional groups suitable for transformation under suitable conditions to provide methanol or methoxide, and functional groups suitable for transformation under suitable conditions to provide formaldehyde or formyl. Functional groups particularly suitable for transformation under suitable conditions to provide methanol or methoxide include those described herein with reference to moiety [X—O—R²]ₙ in Structural Formula I, for example, methyl ortho esters and geminal dimethoxy. Functional groups particularly suitable for transformation under suitable conditions to provide formaldehyde or formyl include hydrolyzable formyl.

In some embodiments, a methanol prodrug comprises a residue (e.g., a residue of a metabolite, amino acid, polyol, food additive and/or GRAS substance) covalently modified with [X—O—R²]n, as that moiety is described herein (e.g., in any of the numbered embodiments or aspects thereof). In some aspects, [X—O—R²] is independently selected from methyl ester and methoxy, provided that when [X—O—R²] is methoxy, there are at least two occurrences (e.g., two or three occurrences) of [X—O—R²] attached to a single carbon atom of the residue. In some aspects, [X—O—R²] is independently selected from methyl ester and methoxy, provided that when [X—O—R²] is methoxy, there are two or three occurrences of [X—O—R²] attached to a single carbon atom of the residue, such that the two or three occurrences of [X—O—R²] attached to a single carbon atom of the residue, taken together, form a geminal dimethoxy or methyl ortho ester, respectively.

In some embodiments, a methanol prodrug comprises a residue (e.g., a residue of a metabolite, amino acid, polyol, food additive and/or GRAS substance) comprising one or more (e.g., from one to five; from two to five; 1, 2 or 3) methyl ortho esters, as that term is described herein (e.g., in any of the numbered embodiments or aspects thereof, such as those pertaining to Structural Formula II). In some aspects, the methanol prodrug further comprises one or more (e.g., one or two; one) geminal dimethoxy, as that term is described herein.

In some embodiments, a methanol prodrug comprises a residue (e.g., a residue of a metabolite, amino acid, polyol, food additive and/or GRAS substance) comprising one or more (e.g., 1, 2 or 3; 1) geminal dimethoxy, as that term is described herein. In some aspects, the methanol prodrug further comprises one or more (e.g., 1, 2 or 3; 1; 2; 3) methyl esters, as that term is described herein.

In some embodiments, the residue is a residue of formic acid, pyruvic acid, oxoglutaric acid (e.g., 2-oxoglutaric acid), oxalosuccinic acid, malonic acid, citric acid, 1-hydroxypropane-1,2,3-tricarboxylic acid, succinic acid, oxaloacetic acid, glycine, glutamic acid, aspartic acid, alanine, trichloroacetic acid or trifluoroacetic acid. In some embodiments, the residue is a residue of glycine, citric acid, lactic acid, formic acid or carbonic acid. In some embodiments, the residue is a residue of lactic acid, pyruvic acid, malic acid, acetic acid, glycine, alanine, oxoglutaric acid (e.g., 2-oxoglutaric acid) or oxaloacetic acid.

In some embodiments, a molecule of prodrug (e.g., methanol prodrug) can provide two or more (e.g., from 2 to 25, inclusive, from 2 to 15, inclusive, from 2 to 10, inclusive, from 2 to 8, inclusive, from 2 to 6, inclusive, from 2 to 5, inclusive, 2, 3, 4, 5 or 6) 1C units suitable for use in 1C metabolism in a subject per molecule of prodrug. In some embodiments, a methanol prodrug provides from 1 to 25, inclusive, e.g., from 1 to 15, inclusive, from 1 to 10, inclusive, from 2 to 10, inclusive, from 1 to 8, inclusive, from 1 to 6, inclusive, from 1 to 5, inclusive, 1, 2, 3, 4, 5 or 6 moles of methanol or methoxide per mole of compound. In some embodiments, a prodrug (e.g., methanol prodrug) provides from 1 to 10, inclusive, e.g., from 1 to 10, inclusive, from 1 to 8, inclusive, from 1 to 6, inclusive, from 1 to 5, inclusive, 1, 2, 3, 4, 5 or 6 moles of formaldehyde or formyl per mole of compound. In some embodiments, a methanol prodrug provides at least one (e.g., from 1 to 5, inclusive, from 1 to 4, inclusive, from 1 to 3, inclusive, 1 or 2, 1, 2, 3, 4 or 5) mole of methanol or methoxide per mole of compound, and at least one (e.g., from 1 to 5, inclusive, from 1 to 4, inclusive, from 1 to 3, inclusive, 1 or 2, 1, 2, 3, 4 or 5) mole of formaldehyde or formyl per mole of compound.

In some embodiments, a molecule of prodrug (e.g., methanol prodrug) has a methanol density of greater than 20%, e.g., greater than 25%, greater than 30%, greater than 40%, greater than 50%, greater than 60% or greater than 70%. Methanol density can be calculated by dividing the number of methanol or methoxide units a molecule of prodrug can provide by the sum of the number of methanol or methoxide units a molecule of prodrug can provide and the total number of remaining non-hydrogen atoms in the molecule of prodrug. Thus, for example, the methanol density of

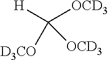

is the quotient of 3/(3+1) or 75%. For example, the methanol density of

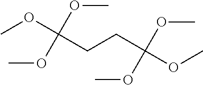

is the quotient of 6/(6+4)=60%. For example, the methanol density of

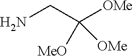

is the quotient of 3/(3+3)=50%.

A first embodiment is a compound represented by the following structural formula:

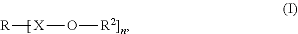

or a pharmaceutically acceptable salt thereof, wherein:
each X is independently —C(O)—, —Y—C(O)—, —C(O)—Y—C(O)—, —Y—CH₂—, —C(O)—Y—CH₂—, —S(O)₂—, —S(O)₂—O—CH₂— or a covalent bond;
   each Y is independently O or N(R¹);
   each R¹ is independently —H, formyl, —CO₂H, —CO₂R², —CH₂—OR², —CO₂(C₂-C₅)alkyl or —CO₂CH₂-(C₆-C₁₅)aryl; or
R is —H, -D, —O*CH₃, —O*CD₃, —O*CH₂D or —O*CHD₂, or (C₁-C₂₅)aliphatic, (C₁-C₂₅)heteroaliphatic or (C₆-C₂₅)aryl substituted with -(R¹⁰)ₘ;
   each R¹⁰ is independently halo, hydroxy, (C₁-C₅)alkoxy, —CO₂H, —CO₂(C₂-C₅)alkyl, —O-formyl or —NR¹¹R¹², or oxo;
   m is 0, 1, 2, 3, 4 or 5;
   R¹¹ and R¹² are each independently —H, formyl, —CO₂H, —CO₂(C₂-C₅)alkyl or —CO₂CH₂—(C₆-C₁₅)aryl;
each R² is independently —*CH₃, —*CD₃, —*CH₂D or —*CHD₂;
n is an integer from 1 to 25, inclusive;
each carbon atom indicated with an * is independently ¹²C or 13C;
each carbon atom of a hydrolyzable formyl is independently ¹²C or ¹³C; and
each hydrogen atom of a hydrolyzable formyl is H or D,
provided that when X is a covalent bond, there are at least two occurrences of X attached to a single carbon atom of R which are a covalent bond.

In a first aspect of the first embodiment, if n is 1, then R is —H, -D, —O*CH₃, —O*CD₃, —O*CH₂D or —O*CHD₂, or the compound contains at least one hydrolyzable formyl. Values for the variables are as described in the first embodiment.

In a second aspect of the first embodiment, each Y is O. Values for the remaining variables are as described in the first embodiment, or first aspect thereof.

In a third aspect of the first embodiment, each Y is N(R¹). Values for the remaining variables are as described in the first embodiment, or first or second aspect thereof.

In a fourth aspect of the first embodiment, each R¹ is independently formyl or —CO₂CH₃. Values for the remaining variables are as described in the first embodiment, or first through third aspects thereof.

In a fifth aspect of the first embodiment, R is (C₁-C₁₅)alkyl substituted with (R¹⁰)ₘ. Values for the remaining variables are as described in the first embodiment, or first through fourth aspects thereof.

In a sixth aspect of the first embodiment, R is (C₁-C₁₀)alkyl substituted with (R¹⁰)ₘ. Values for the remaining variables are as described in the first embodiment, or first through fifth aspects thereof.

In a seventh aspect of the first embodiment, R is (C₁-C₅)alkyl substituted with (R¹⁰)ₘ. Values for the remaining variables are as described in the first embodiment, or first through sixth aspects thereof.

In an eighth aspect of the first embodiment, R is (C₁-C₁₅)alkylether substituted with (R¹⁰)ₘ. Values for the remaining variables are as described in the first embodiment, or first through seventh aspects thereof.

In a ninth aspect of the first embodiment, R is a residue of a metabolite (e.g., citric acid, isocitric acid, aconitic acid, alpha-ketoglutaric acid, fumaric acid, malic acid, succinic acid, lactic acid or pyruvic acid), an amino acid (e.g., glycine, alanine, lysine, aspartic acid, glutamic acid, serine, threonine, asparagine or glutamine), a polyol (e.g., glycerol, erythritol, xylitol, sorbitol, ribose, 2-deoxyribose, fructose, glucose, galactose, mannose, allose, altrose, gulose, idose, talose, xylose, maltitol, isomalt or sucrose) or a food additive (e.g. tartaric acid). Values for variable R and the remaining variables are as described in the first embodiment, or first through eighth aspects thereof.

In a tenth aspect of the first embodiment, R is a metabolite residue. Values for variable R and the remaining variables are as described in the first embodiment, or first through eighth aspects thereof.

In an eleventh aspect of the first embodiment, R is a residue of a metabolite selected from citric acid, isocitric acid, aconitic acid, alpha-ketoglutaric acid, fumaric acid, malic acid, succinic acid, lactic acid or pyruvic acid. Values for variable R and the remaining variables are as described in the first embodiment, or first through eighth aspects thereof.

In a twelfth aspect of the first embodiment, R is an amino acid residue. Values for variable R and the remaining variables are as described in the first embodiment, or first through eighth aspects thereof.

In a thirteenth aspect of the first embodiment, R is a residue of an amino acid selected from glycine, alanine, lysine, aspartic acid, glutamic acid, serine, threonine, asparagine or glutamine. Values for variable R and the remaining variables are as described in the first embodiment, or first through eighth aspects thereof.

In a fourteenth aspect of the first embodiment, R is a polyol residue. Values for variable R and the remaining variables are as described in the first embodiment, or first through eighth aspects thereof.

In a fifteenth aspect of the first embodiment, R is a residue of a polyol selected from glycerol, erythritol, xylitol, sorbitol, ribose, 2-deoxyribose, fructose, glucose, galactose, mannose, allose, altrose, gulose, idose, talose, xylose, maltitol, isomalt or sucrose. Values for variable R and the remaining variables are as described in the first embodiment, or first through eighth aspects thereof.

In a sixteenth aspect of the first embodiment, R is a food additive residue. Values for variable R and the remaining variables are as described in the first embodiment, or first through eighth aspects thereof.

In a seventeenth aspect of the first embodiment, R is a residue of tartaric acid. Values for variable R and the remaining variables are as described in the first embodiment, or first through eighth aspects thereof.

In an eighteenth aspect of the first embodiment, R is —H or -D. Values for the remaining variables are as described in the first embodiment, or first through seventeenth aspects thereof.

In a nineteenth aspect of the first embodiment, R is —O*CH₃ or —O*CD₃. Values for the remaining variables are as described in the first embodiment, or first through eighteenth aspects thereof.

In a twentieth aspect of the first embodiment, each R¹⁰ is independently —CO₂H, —O-formyl or —NR¹¹R¹². Values for the remaining variables are as described in the first embodiment, or first through nineteenth aspects thereof.

In a twenty-first aspect of the first embodiment, m is 0, 1, 2 or 3. Values for the remaining variables are as described in the first embodiment, or first through twentieth aspects thereof.

In a twenty-second aspect of the first embodiment, m is 0. Values for the remaining variables are as described in the first embodiment, or first through twenty-first aspects thereof.

In a twenty-third aspect of the first embodiment, R¹¹ and R¹² are each independently H, formyl or —CO₂(C₂-C₅)alkyl. Values for the remaining variables are as described in the first embodiment, or first through twenty-second aspects thereof.

In a twenty-fourth aspect of the first embodiment, each R² is independently —*CH₃ or —*CD₃. Values for the remaining variables are as described in the first embodiment, or first through twenty-third aspects thereof.

In a twenty-fifth aspect of the first embodiment, each R² is —*CH₃. Values for the remaining variables are as described in the first embodiment, or first through twenty-fourth aspects thereof.

In a twenty-sixth aspect of the first embodiment, each R² is —*CD₃. Values for the remaining variables are as described in the first embodiment, or first through twenty-fifth aspects thereof.

In a twenty-seventh aspect of the first embodiment, n is an integer from 1 to 8, inclusive. Values for the remaining variables are as described in the first embodiment, or first through twenty-sixth aspects thereof.

In a twenty-eighth aspect of the first embodiment, n is an integer from 2 to 10, inclusive. Values for the remaining variables are as described in the first embodiment, or first through twenty-seventh aspects thereof.

In a twenty-ninth aspect of the first embodiment, n is an integer from 1 to 5, inclusive. Values for the remaining variables are as described in the first embodiment, or first through twenty-eighth aspects thereof.

In a thirtieth aspect of the first embodiment, each carbon atom indicated with an asterisk is ¹²C. Values for the remaining variables are as described in the first embodiment, or first through twenty-ninth aspects thereof.

In a thirty-first aspect of the first embodiment, each carbon atom indicated with an asterisk is ¹³C. Values for the remaining variables are as described in the first embodiment, or first through thirtieth aspects thereof.

In a thirty-second aspect of the first embodiment, each carbon atom of a hydrolyzable formyl is ¹²C. Values for the variables are as described in the first embodiment, or first through thirty-first aspects thereof.

In a thirty-third aspect of the first embodiment, each carbon atom of a hydrolyzable formyl is ¹³C. Values for the variables are as described in the first embodiment, or first through thirty-first aspects thereof.

In a thirty-fourth aspect of the first embodiment, each hydrogen atom of a hydrolyzable formyl is H. Values for the variables are as described in the first embodiment, or first through thirty-third aspects thereof.

In a thirty-fifth aspect of the first embodiment, each hydrogen atom of a hydrolyzable formyl is D. Values for the variables are as described in the first embodiment, or first through thirty-third aspects thereof.

In a thirty-sixth aspect of the first embodiment, R is H, D, —O*CH₃ or —O*CD₃. Values for the remaining variables are as described in the first embodiment, or first through thirty-fifth aspects thereof.

In a thirty-seventh aspect of the first embodiment, each X is independently —C(O)—, —Y—C(O)—, —C(O)—Y—C(O)—, —Y—CH₂—, —C(O)—Y—CH₂— or a covalent bond. Values for the remaining variables are as described in the first embodiment, or first through thirty-sixth aspects thereof.

In a thirty-eighth aspect of the first embodiment, each X is independently —C(O)—, —Y—C(O)—, —C(O)—Y—C(O)—, —Y—CH₂— or —C(O)—Y—CH₂—. Values for the remaining variables are as described in the first embodiment, or first through thirty-seventh aspects thereof.

In a thirty-eighth aspect of the first embodiment, R is —H, -D, —O*CH₃, —O*CD₃, -O*CH₂D or —O*CHD₂, or (C₁-C₂₅)aliphatic or (C₁-C₂₅)heteroaliphatic substituted with -(R¹⁰)ₘ. Values for the variables are as described in the first embodiment, or first through thirty-eighth aspects thereof.

In a thirty-ninth aspect of the first embodiment, R is (C₁-C₂₅)aliphatic or (C₁-C₂₅)heteroaliphatic substituted with -(R¹⁰)ₘ. Values for the variables are as described in the first embodiment, or first through thirty-eighth aspects thereof.

In a fortieth aspect of the first embodiments, each R¹⁰ is independently halo, hydroxy, (C₁-C₅)alkoxy, —CO₂H, —CO₂(C₂-C₅)alkyl, —O-formyl or —NR¹¹R¹². Values for the remaining variables are as described in the first embodiment, or first through thirty-ninth aspects thereof.

In a forty-first embodiment, R is —H, -D, —O*CH₃, —O*CD₃, —O*CH₂D or -O*CHD₂, or (C₁-C₁₅)alkyl or (C₁-C₁₅)alkylether substituted with -(R¹⁰)ₘ. Values for the remaining variables are as described in the first embodiment, or first through fortieth aspects thereof.

In a forty-second aspect of the first embodiment, n is an integer from 1 to 10, inclusive. Values for the remaining variables are as described in the first embodiment, or first through forty-first aspects thereof.

In a forty-third aspect of the first embodiment, R is a residue of an endogenous carboxylic acid. Values for the remaining variables are as described in the first embodiment, or first through forty-second aspects thereof.

In a forty-fourth aspect of the first embodiment, each X is independently —C(O)— or a covalent bond. Values for the remaining variables are as described in the first embodiment, or first through forty-third aspects thereof.

In a forty-fifth aspect of the first embodiment, each R¹⁰ is independently halo, hydroxy, (C₁-C₅)alkoxy, —CO₂H, —CO₂(C₂-C₅)alkyl, or oxo. Values for the remaining variables are as described in the first embodiment, or first through forty-fourth aspects thereof.

A second embodiment is a compound of Structural Formula I, or a pharmaceutically acceptable salt thereof, wherein:
each X is independently —C(O)—, —Y—C(O)—, —C(O)—Y—C(O)—, —Y—CH₂—, —C(O)—Y—CH₂— or a covalent bond;
   each Y is independently O or N(R¹);
   each R¹ is independently —H, formyl, —CO₂H, —CO₂R², —CH₂—OR², —CO₂(C₂-C₅)alkyl or —CO₂CH₂—(C₆-C₁₅)aryl; or
R is —H, -D, —O*CH₃, —O*CD₃, —O*CH₂D or —O*CHD₂, or (C₁-C₁₅)alkyl or (C₁-C₁₅)alkylether substituted with -(R¹⁰)ₘ;
   each R¹⁰ is independently halo, hydroxy, (C₁-C₅)alkoxy, —CO₂H, —CO₂(C₂-C₅)alkyl, —O-formyl or —NR¹¹R¹²;
   m is 0, 1, 2, 3, 4 or 5;
   R¹¹ and R¹² are each independently —H, formyl, —CO₂H, —CO₂(C₂-C₅)alkyl or —CO₂CH₂—(C₆-C₁₅)aryl;
each R² is independently —*CH₃, —*CD₃, —*CH₂D or —*CHD₂;
n is an integer from 1 to 10, inclusive;
each carbon atom indicated with an * is independently ¹²C or 13C;
each carbon atom of a hydrolyzable formyl is independently ¹²C or ¹³C; and each hydrogen atom of a hydrolyzable formyl is H or D, provided:
(i) when X is a covalent bond, there are at least two occurrences of X attached to a single carbon atom of R which are a covalent bond, and
(ii) if n is 1, then R is —H, -D, —O*CH₃, —O*CD₃, —O*CH₂D or —O*CHD₂, or the compound contains at least one hydrolyzable formyl.
   Alternative values for the variables are as described in the first embodiment, or any aspect thereof.

A third embodiment is a compound of Structural Formula II:

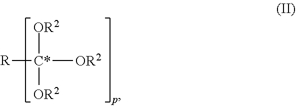

or a pharmaceutically acceptable salt thereof, wherein:
R is H or D, or (C₁-C₁₅)alkyl substituted with -(R²⁰)ₘ;
   each R²⁰ is independently halo, hydroxy, —OR², (C₂-C₅)alkoxy, —CO₂H, —CO₂R², —CO₂(C₂-C₅)alkyl, —O-formyl or —NR²¹R²²;
   m is 0, 1, 2, 3, 4 or 5;
   R²¹ and R²² are each independently —H, formyl, —CO₂H, —CO₂R², —CO₂(C₂-C₅)alkyl or —CO₂CH₂—(C₆-C₁₅)aryl;
each R² is independently —*CH₃, —*CD₃, —*CH₂D or —*CHD₂;
p is an integer from 1 to 10, inclusive;
each carbon atom indicated with an * is independently ¹²C or 13C;
each carbon atom of a hydrolyzable formyl is independently ¹²C or ¹³C; and
each hydrogen atom of a hydrolyzable formyl is H or D.
   Alternative values for the variables (e.g., R, m) are as described in the first or second embodiment, or any aspect thereof.

In a first aspect of the third embodiment, R is H or D. Values for the remaining variables are as described in the first or second embodiment, or any aspect thereof, or the third embodiment.

In a second aspect of the third embodiment, R is (C₁-C₁₅)alkyl substituted with -(R²⁰)ₘ. Values for the remaining variables are as described in the first or second embodiment, or any aspect thereof, or the third embodiment, or first aspect thereof.

In a third aspect of the third embodiment, R is (C₁-C₁₀)alkyl substituted with (R²⁰)ₘ. Values for the remaining variables are as described in the first or second embodiment, or any aspect thereof, or the third embodiment, or first or second aspect thereof.

In a fourth aspect of the third embodiment, R is (C₁-C₅)alkyl substituted with (R²⁰)ₘ. Values for the remaining variables are as described in the first or second embodiment, or any aspect thereof, or the third embodiment, or first through third aspects thereof.

In a fifth aspect of the third embodiment, each R²⁰ is independently halo, hydroxy, —O-R² or —NR²¹R²². Values for the remaining variables are as described in the first or second embodiment, or any aspect thereof, or the third embodiment, or first through fourth aspects thereof.

In a sixth aspect of the third embodiment, each R²⁰ is independently fluoro, chloro, hydroxy, —O—R² or —NH₂. Values for the remaining variables are as described in the first or second embodiment, or any aspect thereof, or the third embodiment, or first through fifth aspects thereof.

In a seventh aspect of the third embodiment, R²¹ and R²² are each independently H, formyl or —CO₂R². Values for the remaining variables are as described in the first or second embodiment, or any aspect thereof, or the third embodiment, or first through sixth aspects thereof.

In an eighth aspect of the third embodiment, p is an integer from 1 to 5, inclusive. Values for the remaining variables are as described in the first or second embodiment, or any aspect thereof, or the third embodiment, or first through seventh aspects thereof.

In a ninth aspect of the third embodiment, p is an integer from 2 to 5, inclusive. Values for the remaining variables are as described in the first or second embodiment, or any aspect thereof, or the third embodiment, or first through eighth aspects thereof.

A fourth embodiment is a compound of Structural Formula I or II, or a pharmaceutically acceptable salt thereof, wherein R is a residue described herein (e.g., a residue of a metabolite, amino acid, polyol, food additive, GRAS substance or carboxylic acid (e.g., endogenous carboxylic acid)). Values for the remaining variables are as described in the first through third embodiments, or any aspect thereof.

In a further aspect of any of the aforementioned embodiments or aspects thereof, the compound is not dimethyladipate, trimethylorthoformate, trimethylorthoacetate or tetramethoxymethane, or a salt of the foregoing.

In a further aspect of any of the aforementioned embodiments or aspects thereof, the compound is not trimethylorthoformate, trimethoxymethane-d, trimethoxymethane-¹³C or (bis(methoxy-d₃)methoxy-d)methane-d₃, or a salt of the foregoing.

In a further aspect of any of the aforementioned embodiments or aspects thereof, the compound is not trimethylorthoacetate, 1,1,1,2,2-pentamethoxypropane, 1,1,1,3,3,3-hexamethoxypropane, 1,1,1,4,4,4-hexamethoxybutane, 2,2,2-trimethoxyethan-1-amine, 1,1,1-trimethoxypropan-2-amine, 1,1,1-trichloro-2,2,2-trimethoxyethane or 1,1,1-trifluoro-2,2,2-trimethoxyethane, or a salt of the foregoing.

In a further aspect of any of the aforementioned embodiments or aspects thereof of a compound of Structural Formula I, R is not —H or -D when X is —C(O)— and n is 1.

Examples of compounds of Structural Formula I derived from glycine include:

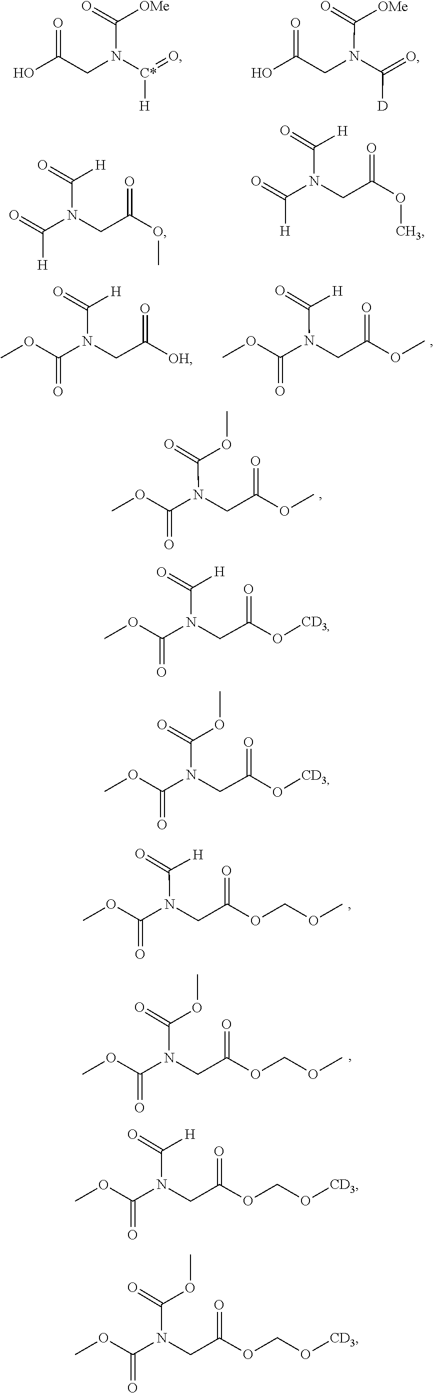

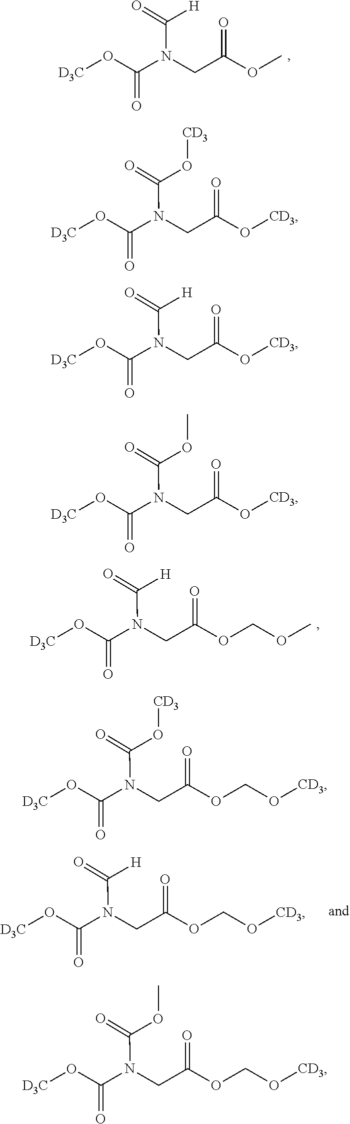

or a pharmaceutically acceptable salt of the foregoing.

Examples of compounds of Structural Formula I derived from citric acid include:

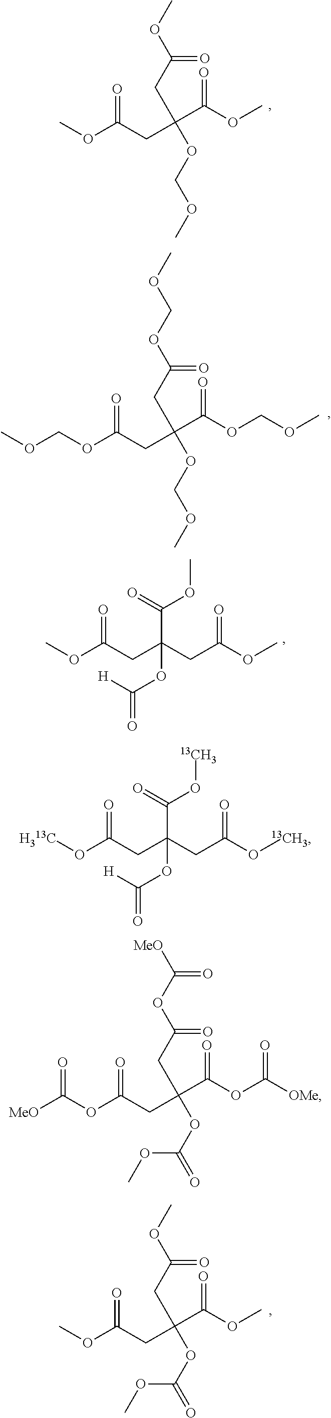

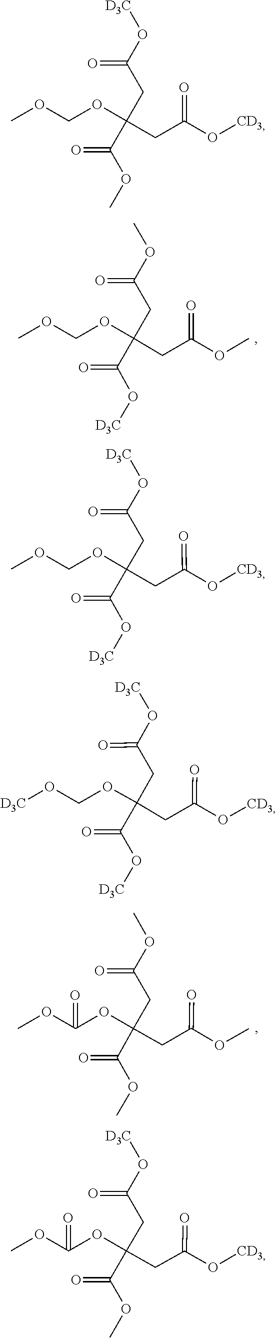

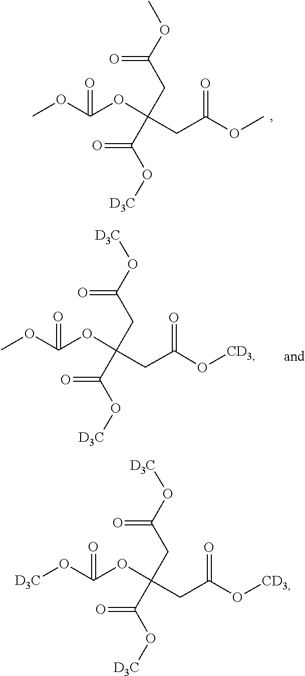

or a pharmaceutically acceptable salt of the foregoing. Examples of compounds of Structural Formula I derived from lactic acid include:

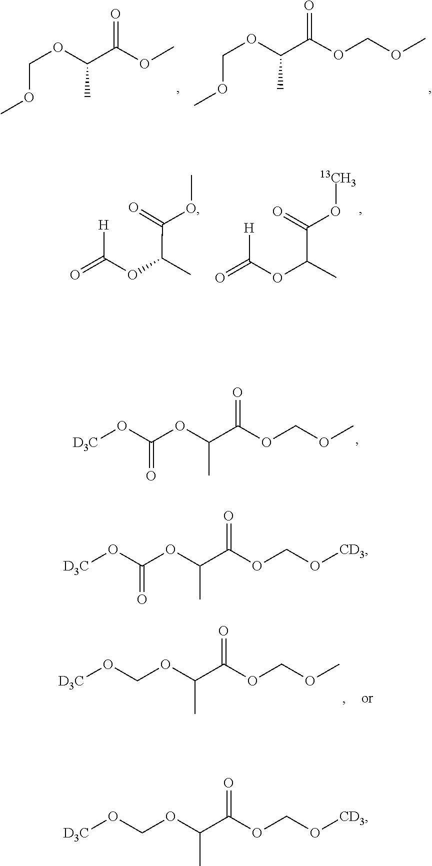

or a pharmaceutically acceptable salt of the foregoing. Examples of compounds of Structural Formula I derived from formic acid include:

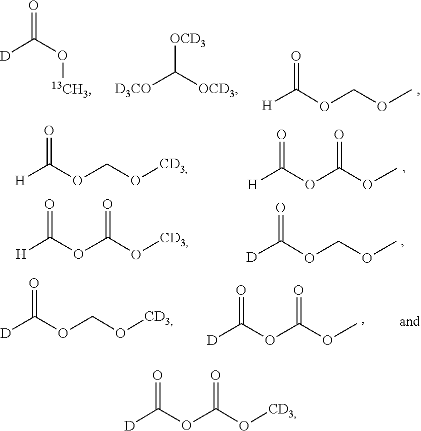

or a pharmaceutically acceptable salt of the foregoing. Examples of compounds of Structural Formula I derived from carbonic acid include:

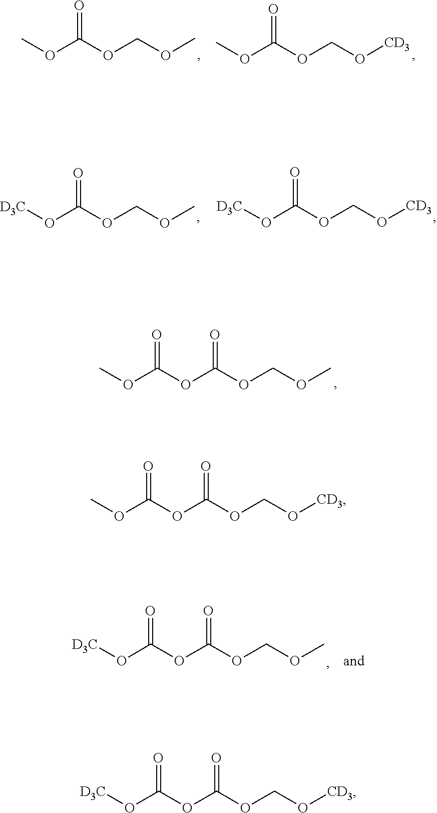

or a pharmaceutically acceptable salt of the foregoing. Other compounds of Structural Formula I include:

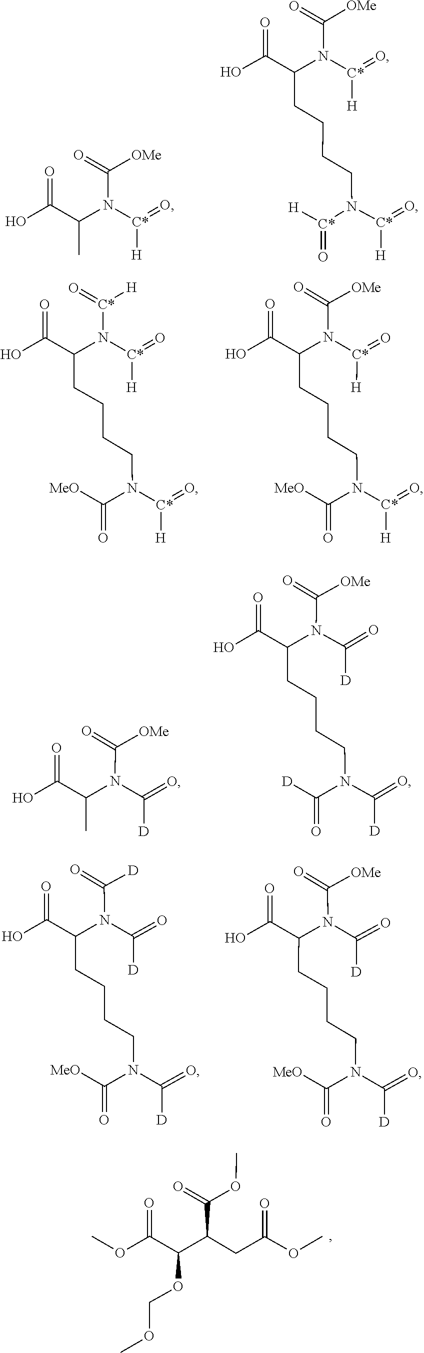

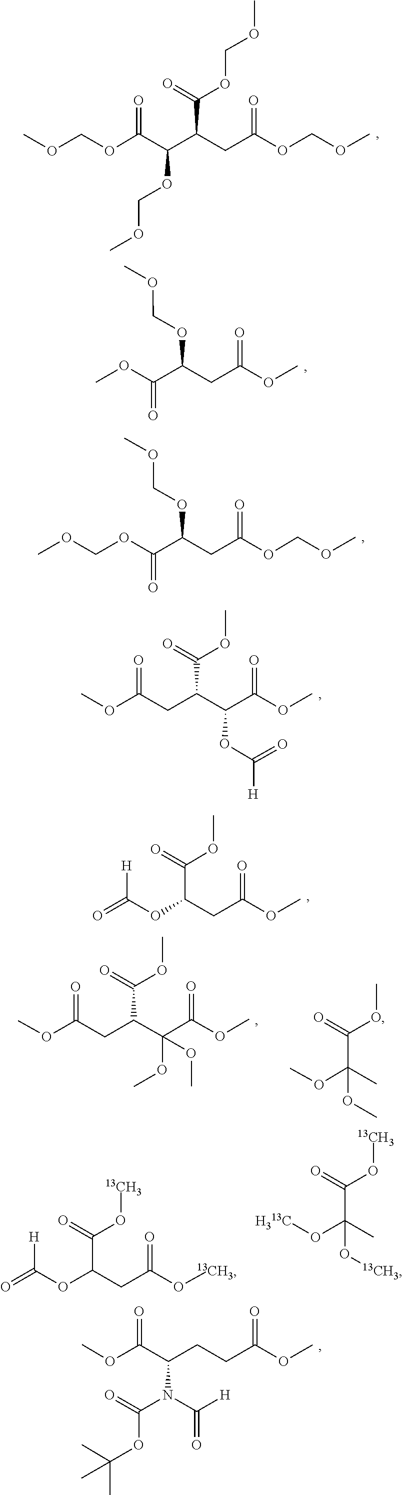

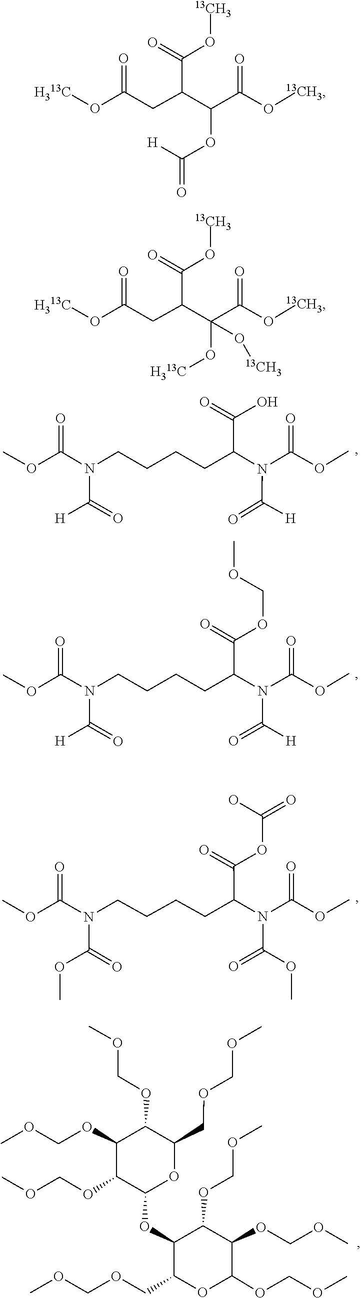

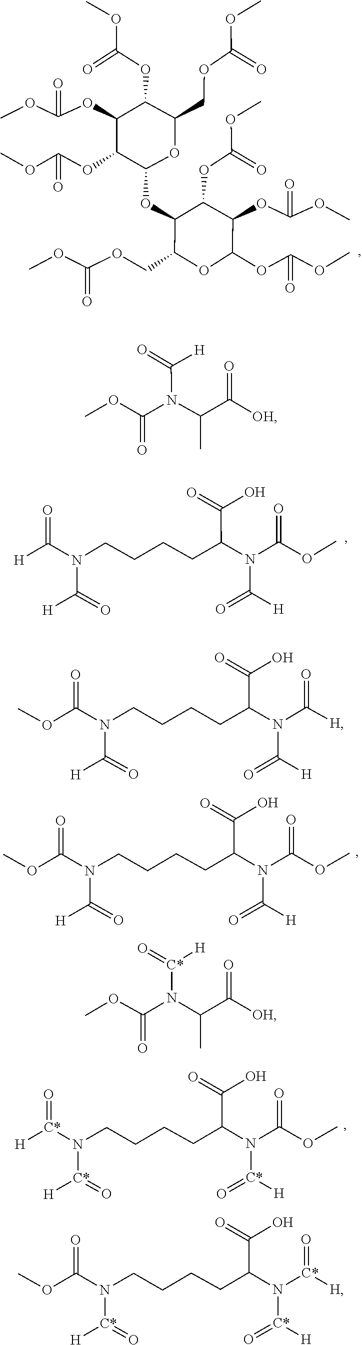

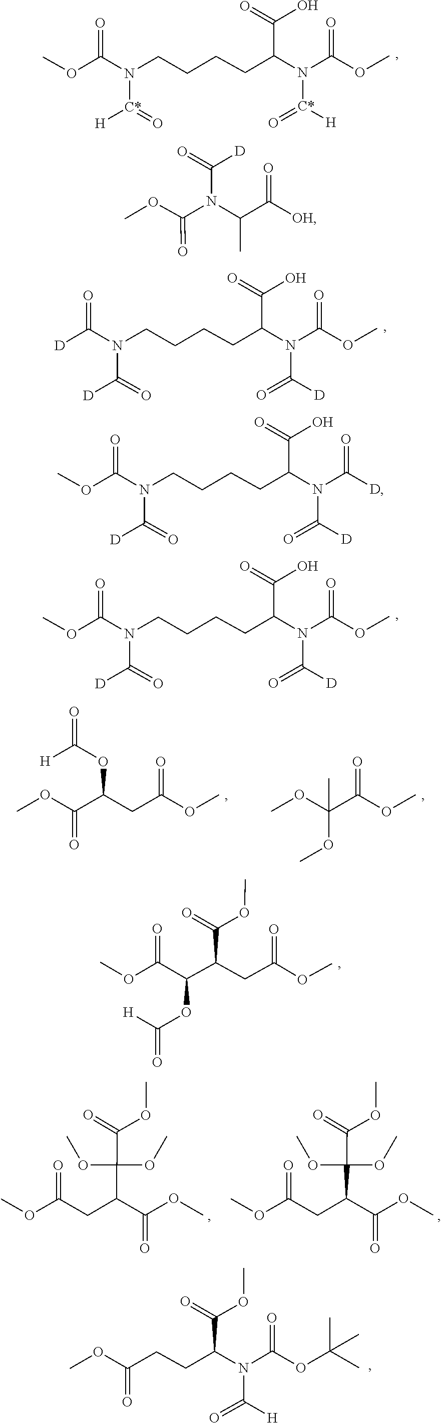

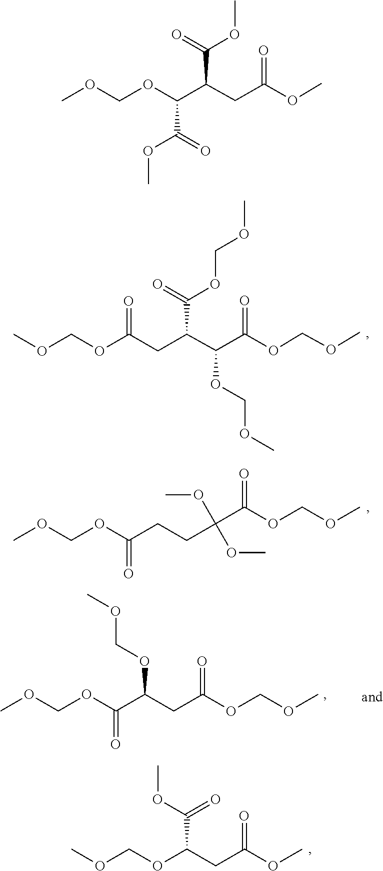

or a pharmaceutically acceptable salt of the foregoing, wherein each carbon marked with an asterisk is ¹³C. Yet other compounds of Structural Formula I include

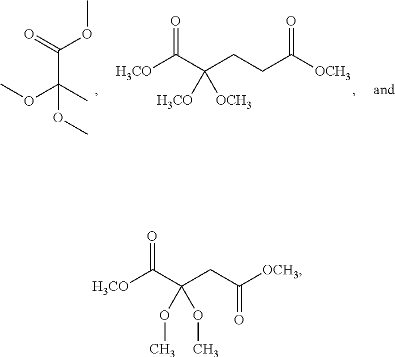

or a pharmaceutically acceptable salt of the foregoing.

Examples of methyl ortho ester compounds of Structural Formula I (e.g. Structural Formula II) include

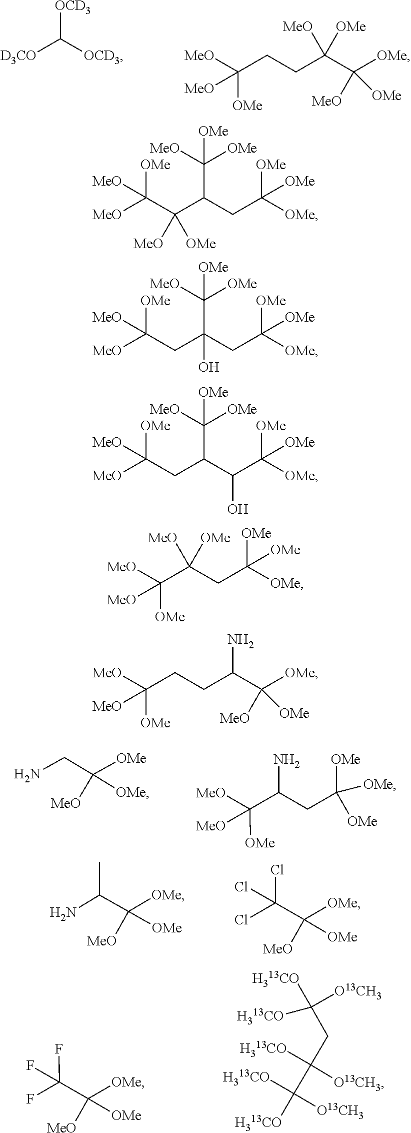

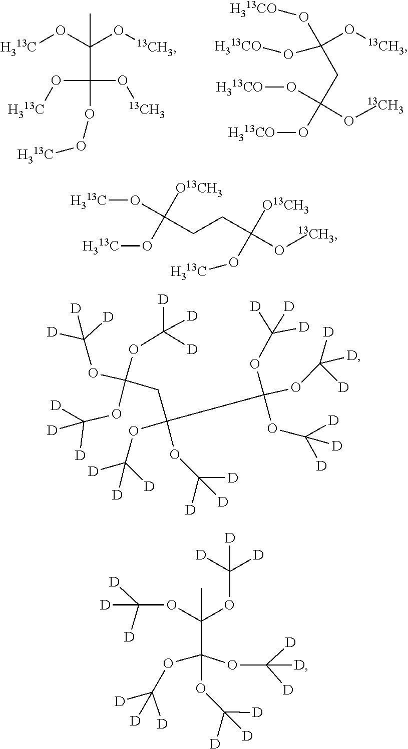

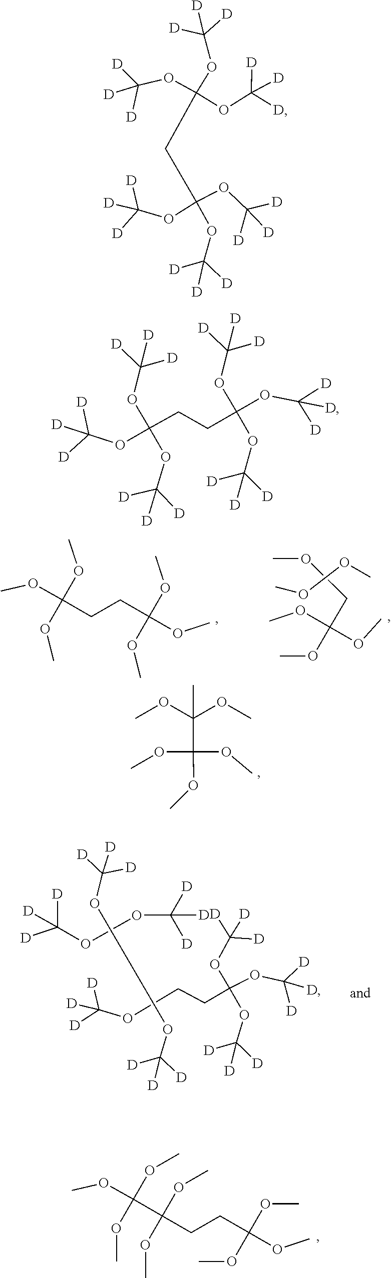

or a pharmaceutically acceptable salt of the foregoing. Methyl ortho esters of Structural Formula I (e.g., Structural Formula II) can be derived, e.g., from formic acid, pyruvic acid, oxoglutaric acid (e.g., 2-oxoglutaric acid), oxalosuccinic acid, malonic acid, citric acid, 1-hydroxypropane-1,2,3-tricarboxylic acid, succinic acid, 2-oxosuccinic acid, glycine, glutamic acid, aspartic acid, alanine, trichloroacetic acid and trifluoroacetic acid.

Methods of making compounds of Structural Formula I, or a pharmaceutically acceptable salt thereof, are within the abilities of a person skilled in the art and described herein. For example, Rogozhin, S. V., et al., “Synthesis of the orthomethyl ester of glycine,” Izvestdya A kademii Nauk SSSR, Seilya Khimicheskaya 4, 956 (1970) describes the synthesis of the orthomethyl ester of glvcire

### Pharmaceutical Compositions, Pharmaceutical Combinations, Kits

Typically, for administration to a subject, a compound of the disclosure is formulated with one or more pharmaceutically acceptable carriers. The disclosure provides such compositions, including pharmaceutical compositions. Thus, one embodiment is a composition (e.g., pharmaceutical composition) comprising a compound of the disclosure and a pharmaceutically acceptable carrier. The compositions described herein can be used in the methods described herein, e.g., to supply a compound of the disclosure for administration to a subject.

Compositions described herein and, hence, compounds of the disclosure, may be administered orally, parenterally (including subcutaneously, intramuscularly, intravenously and intradermally), by inhalation spray, topically, rectally, nasally, buccally, vaginally or via an implanted reservoir. The terms “parenteral” and “parenterally,” as used herein, include subcutaneous, intracutaneous, intravenous, intramuscular, intraocular, intravitreal, intra-articular, intra-arterial, intra-synovial, intrasternal, intrathecal, intralesional, intrahepatic, intraperitoneal, intralesional and intracranial injection or infusion techniques. In some embodiments, a composition described herein is administrable intravenously and/or intraperitoneally. In some embodiments, a composition described herein is administrable orally. Preferably, a composition described herein is administered orally, subcutaneously, intraperitoneally or intravenously.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound of the disclosure can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another aspect, a compound of the disclosure can be provided in an extended (or “delayed” or “sustained”) release composition. This delayed-release composition comprises the compound of the disclosure and a delayed-release component. Such a composition allows targeted release of the compound, for example, into the lower gastrointestinal tract, for example, into the small intestine, the large intestine, the colon and/or the rectum. In certain aspects, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition can provide controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously, e.g., in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, dextrose, water, Ringer's solution, lactated Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

Compositions described herein can also be administered in the form of suppositories for rectal administration. These can be prepared by mixing a compound of the disclosure with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound described herein include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. Suitable carriers also include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

Other pharmaceutically acceptable carriers that can be used in the compositions described herein include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

In some embodiments, a composition described herein further includes one or more additional therapeutic agents, e.g., for use in combination with a compound of the disclosure as, for example, when the compound of the disclosure and the one or more additional therapeutic agents are to be co-administered.

Some embodiments provide a combination (e.g., pharmaceutical combination) comprising a compound of the disclosure (e.g., a composition described herein comprising a compound of the disclosure) and one or more additional therapeutic agents (e.g., one or more compositions comprising one or more additional therapeutic agents). Such combinations are particularly useful as, for example, when the compound of the disclosure and the one or more additional therapeutic agents are to be administered separately. In a combination provided herein, the compound of the disclosure and the one or more additional therapeutic agents can be administrable by the same route of administration or by different routes of administration.

Some embodiments provide a kit comprising a compound of the disclosure (e.g., a composition described herein comprising a compound of the disclosure) and an additional therapeutic agent(s) (e.g., a composition comprising an additional therapeutic agent(s)). In one embodiment, the kit comprises a therapeutically effective amount of the compound of the disclosure to treat a disease, disorder or condition described herein, and a therapeutically effective amount of the one or more additional therapeutic agents to treat the disease, disorder or condition. In some embodiments, the kit further comprises written instructions for administering the compound of the disclosure and/or the additional agent(s) to a subject to treat a disease, disorder or condition described herein.

Additional therapeutic agents for use in the compositions, combinations and/or kits provided herein include any of those discussed herein with respect to combination therapies. In some embodiments, the additional therapeutic agent is a checkpoint inhibitor, such as a PD-1 and/or PD-L1 inhibitor. In some embodiments, the checkpoint inhibitor is an inhibitor of CTLA-4, PD-1, PD-L1 or LAG-3. In some embodiments, the checkpoint inhibitor is a PD-1 inhibitor, for example, nivolumab, pembrolizumab or cemiplimab. In some embodiments, the checkpoint inhibitor is a PD-L1 inhibitor, for example, avelumab, durvalumab or atezolizumab.

The compositions described herein can be provided in unit dosage form. The amount of a compound of the disclosure or other therapeutic agent that can be combined with the carrier materials to produce a composition in a unit dosage form will vary depending, for example, upon the subject treated, the particular mode of administration and the activity of the agent employed. Preferably, compositions should be formulated so that a compound of the disclosure or other therapeutic agent can be administered to a subject receiving the composition at a dosage and/or frequency consistent with those described herein. Typically, a unit dosage form will contain from about 1 mg to about 5,000 mg, from about 10 mg to about 2,500 mg, from about 100 mg to about 1,000 mg, from about 1 mg to about 1000 mg, from about 1 mg to about 500 mg, from about 1 mg to about 250 mg, from about 1 mg to about 150 mg, from about 0.5 mg to about 100 mg, or from about 1 mg to about 50 mg of active ingredient(s).

In some embodiments, the concentration of one or more therapeutic agents provided in a composition is less than 100%, 90%, 80%, 70%, 60%, 50%, 40%, 30%, 20%, 19%, 18%, 17%, 16%, 15%, 14%, 13%, %12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v; and/or greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v. In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Uses and Therapeutic Methods

One-carbon (1C) metabolism is the process by which one-carbon, or single-carbon, units are transferred from one molecule to another. Typically, in 1C metabolism, a carbon unit is transferred from serine or glycine to tetrahydrofolate (TIF) to form methylene-THF. Examples of one-carbon units include methyl (—CH₃), methylene (═CH₂), methenyl (═CH₂—), formyl, formimino (—CH═NH—) and hydroxymethyl (—CH₂OH). Sources of one-carbon units include serine, glycine, histidine, tryptophan, formic acid, 5-formyl-THF, 5-methyl-THF, monomethylglycine, dimethylglycine, glycine betaine, choline and glucose. Formate, in particular, can be used to supply 1C units directly to 1C metabolism, and has been shown to rescue T-cell activity in 1C-starved T-cells, such as tumor-resident or aged T-cells, but is too rapidly eliminated to dose directly.

FIG. 1 depicts liver methanol metabolism. In the liver, methanol is transformed into formic acid/formate in a two-step process. The first step involves oxidation of methanol to formaldehyde by alcohol dehydrogenase (ADH) with concomitant reduction of nicotinamide adenine dinucleotide (NAD⁺) to NADH. In the second step, formaldehyde is further oxidized to formic acid/formate by aldehyde dehydrogenase (ALDH), again with concomitant reduction of NAD⁺ to NADH. The final step in the metabolism of methanol in the liver is the oxidative conversion of formic acid/formate into carbon dioxide by methylenetetrahydrofolate dehydrogenase 1 (MTHFD1)/10-formyltetrahydrofolate dehydrogenase (ALDH1L1). The carbon dioxide thus produced is then exhaled in the breath.

It is shown herein that methanol can provide extended release of formate and synergize with immunotherapy in a mouse model of cancer at levels expected to be at least 20-fold lower than those observed to produce symptomatic toxicity in primates. Without wishing to be bound by any particular theory, it is believed that extended release of formate is provided by methanol because formaldehyde does not accumulate during methanol administration, suggesting that the ALDH oxidation of formaldehyde to formic acid/formate is rapid, while the oxidation of formic acid/formate to carbon dioxide is much slower. Thus, the repository of formic acid/formate produced by the liver during metabolism and, by extension, methanol is also a potential source of one-carbon units.

Thus, provided herein is a method of promoting the survival, proliferation or activation of an immune cell, comprising contacting the immune cell with an effective amount of a compound of the disclosure (e.g., methanol, a methanol prodrug). Some embodiments comprise contacting the immune cell with an effective amount of methanol or deuteromethanol. Some embodiments comprise contacting the immune cell with a prodrug of methanol or a prodrug of deuteromethanol. In some embodiments, the method is conducted in vitro. In other embodiments, the method is conducted in vivo. In some embodiments, therefore, the immune cell is in a subject (e.g., a subject having a disease, disorder or condition described herein). Examples of immune cells include T-cells, natural killer (NK) cells, macrophages, neutrophils, myeloid-derived suppressor cells or dendritic cells. In some embodiments, an immune cell is a T-cell, such as a CD8⁺ T-cell.

Also provided herein is a method of promoting an immune response in a subject in need thereof, comprising administering to the subject a therapeutically effective amount of a compound of the disclosure (e.g., methanol, a methanol prodrug).

Also provided herein is a method of treating cancer in a subject in need thereof, comprising administering to the subject a therapeutically effective amount of a compound of the disclosure (e.g., methanol, a methanol prodrug).

The cancer can be a solid tumor cancer or a hematological cancer (e.g., a leukemia, a lymphoma or a myeloma). In some embodiments, the cancer is a solid tumor cancer. In some embodiments, the cancer comprises a solid tumor. Examples of solid tumor cancers include breast cancer, colon cancer, colorectal cancer, stomach cancer, ovarian cancer, uterine cancer, bladder cancer, lung cancer, pancreatic cancer, prostate cancer, sarcoma, skin cancer (e.g., melanoma, squamous cell carcinoma), brain cancer, head and neck cancer, lymphoid cancer and liver cancer. Solid tumors may have one or more features selected from poor perfusion, a low NAD⁺/NADH ratio, a low oxygen (O₂) level, or a high lactate level.

In some embodiments, the cancer is a hematologic cancer. Examples of hematologic cancers include leukemia (e.g., acute lymphoblastic leukemia (ALL), acute myeloid leukemia (AML) such as FLT3 inhibitor-resistant AML or AML with high mTORC1 expression and/or activity, chronic lymphocytic leukemia (CLL), chronic myelogenous leukemia (CML), acute monocytic leukemia (AMoL)), lymphoma (e.g., non-Hodgkin's lymphoma or Hodgkin's lymphoma), and multiple myeloma. In certain embodiments, the cancer is a leukemia, preferably a T-cell leukemia, such as T-cell lymphoblastic leukemia. In certain embodiments, the cancer is a B-cell leukemia. In certain embodiments, the cancer is a lymphoma, preferably a T-cell lymphoma. In certain embodiments, the cancer is a B-cell lymphoma, for example, diffuse large B-cell lymphoma or a Burkitt lymphoma.

Specific examples of cancer treatable according to the methods described herein include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. In some embodiments, the cancer is a metastatic cancer.

In some embodiments, the cancer is resistant.

In some embodiments, the cancer is breast cancer, bladder cancer, cervical cancer, colon cancer, head and neck cancer, lymphoma, liver cancer, lung cancer, kidney cancer, skin cancer, stomach cancer, esophageal cancer, rectal cancer, pancreatic cancer, ovarian cancer or a solid tumor that is not able to repair errors in its DNA that occur when the DNA is copied. In some embodiments, the cancer is pancreatic cancer. In some embodiments, the cancer is ovarian cancer.

Also provided herein is a method for enhancing an immunotherapy in a subject in need thereof (e.g., a subject having cancer, including any of the cancers described herein; a subject, such as an aged human, receiving a vaccine), comprising administering to the subject a therapeutically effective amount of a compound of the disclosure (e.g., methanol, a methanol prodrug). Immunotherapies include any of the immunotherapies described herein with respect to combination therapy. In some embodiments, the immunotherapy comprises a checkpoint inhibitor, for example, a PD-1 and/or PD-L1 inhibitor. In some embodiments, the checkpoint inhibitor is an inhibitor of CTLA-4, PD-1, PD-L1 or LAG-3. In some embodiments, the checkpoint inhibitor is a PD-1 inhibitor, for example, nivolumab, pembrolizumab or cemiplimab. In some embodiments, the checkpoint inhibitor is a PD-L1 inhibitor, for example, avelumab, durvalumab or atezolizumab. In some embodiments, the immunotherapy comprises a vaccine.

Also provided herein is a method of treating an immune dysfunction in a subject in need thereof, comprising administering to the subject a therapeutically effective amount of a compound of the disclosure (e.g., methanol, a methanol prodrug).

Some embodiments comprise administering to the subject a therapeutically effective amount of methanol (e.g., deuteromethanol). Some embodiments comprise administering to the subject a therapeutically effective amount of a prodrug of methanol (e.g., a compound of any of the structural formulas described herein, such as Structural Formula I and/or II, or a pharmaceutically acceptable salt thereof).

Also provided herein are methods of promoting an immune response; treating cancer; enhancing an immunotherapy; and/or treating an immune dysfunction in a subject in need thereof, comprising treating the subject with a therapeutically effective amount of methanol (e.g., deuteromethanol) and/or methoxide (e.g., a deuterium isotopologue of methoxide), as, for example, by administering a therapeutically effective amount of methanol or a prodrug of methanol to the subject. In some embodiments, the method is a method of promoting an immune response. In some embodiments, the method is a method of treating cancer. In some embodiments, the method is a method of enhancing an immunotherapy. In some embodiments, the method is a method of treating an immune dysfunction. Some embodiments comprise treating the subject with a therapeutically effective amount of methanol (e.g., deuteromethanol). Some embodiments comprise treating the subject with a therapeutically effective amount of methoxide (e.g., a deuterium isotopologue of methoxide, such as a deuterium and carbon isotopologue of methoxide).

A compound of the disclosure can also be administered in combination with one or more other therapies (e.g., radiation therapy, a chemotherapy, such as a chemotherapeutic agent; an immunotherapy, such as an immunotherapeutic agent) to treat a disease, disorder or condition. When administered “in combination,” the compound of the disclosure can be administered before, after or concurrently with the other therapy(ies) (e.g., radiation therapy, an additional therapeutic agent(s)). When co-administered simultaneously (e.g., concurrently), the compound of the disclosure and another therapeutic agent can be in separate formulations or the same formulation. Alternatively, the compound of the disclosure and another therapeutic agent can be administered sequentially, either at approximately the same time or at different times, as separate compositions. When the compound of the disclosure and the other therapy (e.g., therapeutic agent) are administered as separate formulations or compositions, the compound of the disclosure and the other therapy can be administered by the same route of administration or by different routes of administration. A skilled clinician can determine appropriate timing for administration of each therapy being used in combination (e.g., timing sufficient to allow an overlap of the pharmaceutical effects of the therapies). Typically, a combination therapy will provide beneficial effects of the drug combination in treating the diseases, conditions or disorders described herein.

In some embodiments, a method described herein further comprises administering to the subject (e.g., a therapeutically effective amount of) an additional therapy(ies) (e.g., radiation therapy; an additional therapeutic agent, such as a chemotherapeutic agent, an immunotherapeutic agent, an antibody, such as a monoclonal antibody; a vaccine), e.g., in combination with the compound of the disclosure. In some embodiments, the compound of the disclosure is administered before the additional therapy(ies). In some embodiments, the compound of the disclosure is administered after the additional therapy(ies). In some embodiments, the compound of the disclosure is administered concurrently with the additional therapy(ies).

In some embodiments, a method further comprises administering to the subject radiation therapy (e.g., a therapeutically effective amount of radiation therapy), e.g., proton beam therapy.

In some embodiments, a method further comprises administering to the subject hormone therapy (e.g., a therapeutically effective amount of hormone therapy), e.g., anti-estrogen therapy, androgen deprivation therapy (ADT), such as flutamide, nilutamide, bicalutamide, leuprolide or goserelin, a luteinizing hormone-releasing hormone (LHRH) agonist, an aromatase inhibitor (AI), such as anastrozole, exemestane or letrozole, an estrogen receptor modulator, such as tamoxifen, raloxifene or toremifene.

In some embodiments, e.g., for the treatment of cancer, a method further comprises administering to the subject an immunotherapy (e.g., a therapeutically effective amount of an immunotherapy). Cancer immunotherapy refers to a diverse set of therapeutic strategies designed to induce a subject's own immune system to fight a tumor. Cancer immunotherapy agents include antibodies that inhibit proteins expressed by cancer cells, vaccines and immune cell (e.g., T-cell) infusions. Antibody agents useful for promoting anti-tumor responses include anti-CTLA-4 antibodies (e.g., ipilimumab, tremelimumab), anti-PD-1 antibodies (e.g., pembrolizumab, nivolumab, cemiplimab), anti-PD-L1 antibodies (e.g., atezolizumab, avelumab, durvalumab), anti-PD-L2 antibodies, anti-TIM-3 antibodies, anti-LAG-3 antibodies (e.g., relatlimab), anti-OX40 antibodies and anti-GITR antibodies. In some embodiments, the immunotherapy is an immune checkpoint inhibitor (e.g., a therapeutically effective amount of an immune checkpoint inhibitor). Examples of immune checkpoint inhibitors include inhibitors of CTLA-4 (e.g., ipilimumab, tremelimumab), PD-1 (e.g., nivolumab, pembrolizumab), PD-L1 (e.g., avelumab), PD-L2, TIM-3, LAG-3 (e.g., relatlimab), OX40 and GITR. In some embodiments, the immune checkpoint inhibitor (e.g., for treating a cancer, such as a solid tumor cancer) is an inhibitor of CTLA-4, PD-1, PD-L1 or LAG-3.

In some embodiments, a method further comprises administering to the subject a chemotherapy (e.g., a therapeutically effective amount of a chemotherapy), e.g., comprising one or more chemotherapeutic agents. Examples of chemotherapeutic agents include, for example, antimetabolites (e.g., folic acid, nucleotide analogs, in particular, purine and pyrimidine derivatives); alkylating agents (e.g., cyclophosphamide, mechlorethamine, chlorambucil, melphalan, dacarbazine, temozolomide, thiotepa); anthracyclines (e.g., daunorubicin, doxorubicin, epirubicin, idarubicin, mitoxantrone, valrubicin); taxanes (e.g., paclitaxel, docetaxel, abraxane, taxotere); epothilones; histone deacetylase inhibitors (e.g., vorinostat, romidepsin); topoisomerase inhibitors (e.g., irinotecan, topotecan, etoposide, teniposide, tafluposide); kinase inhibitors (e.g., bortezomib, erlotinib, gefitinib, imatinib, vemurafenib, vismodegib); nucleotide analogs (e.g., azacitidine, azathioprine, capecitabine, cytarabine, doxifluridine, fluorouracil, gemcitabine, hydroxyurea, mercaptopurine, methotrexate, tioguanine); peptide antibiotics (e.g., bleomycin, actinomycin); platinum-based agents (e.g., carboplatin, cisplatin, oxaliplatin); retinoids (e.g., tretinoin, alitretinoin, bexarotene); and vinca alkaloids (e.g., vinblastine, vincristine, vindesine, vinorelbine), as well as their pharmaceutically acceptable salts. Further examples of chemotherapeutic agents include alkylating agents, such as thiotepa and cyclophosphamide (CYTOXAN™); alkyl sulfonates, such as busulfan, improsulfan and piposulfan; aziridines, such as benzodopa, carboquone, meturedopa, and uredopa; ethylenimines and methylamelamines, such as altretamine, triethylenemelamine, trietylenephosphoramide, triethylenethiophosphaoramide and trimethylolomelamine; acetogenins, such as bullatacin and bullatacinone; camptothecins, including the synthetic analogue topotecan; bryostatin; callystatin; CC-1065, including its adozelesin, carzelesin and bizelesin analogues; cryptophycins, such as cryptophycin 1 and cryptophycin 8; dolastatin; duocarmycin, including the synthetic analogues, KW-2189 and CBI-TMI; eleutherobin; pancratistatin; sarcodictyins; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosoureas, such as carmustine, chlorozotocin, fotemustine, lomustine, nimustine, and ranimustine; antibiotics, such as the enediyne antibiotics (e.g., calicheamicin, especially calicheamicin gamma 1 and calicheamicin theta I, see, e.g., Angew Chem. Intl. Ed. Engl. 33:183-186 (1994); dynemicin, such as dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, methotrexate, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, floxuridine, and 5-FU; androgens, such as calusterone, dromostanolone propionate, epitiostanol, mepitiostane, and testolactone; anti-adrenals, such as aminoglutethimide, mitotane, and trilostane; folic acid replenishers, such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; epothilones; etoglucid; gallium nitrate; hydroxyurea; lentinan; lonidamine; maytansinoids, such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes, such as T-2 toxin, verracurin A, roridin A and anguidine; urethan; vindesine; dacarbazine; mannomustine; mitobronitol; mitolactol; pipobroman; gacytosine; arabinoside (“Ara-C”); cyclophosphamide; thiotepa; taxoids, such as paclitaxel (e.g., TAXOL™, Bristol-Myers Squibb Oncology, Princeton, N.J.; nab-paclitaxel, such as the nanoparticle albumin-bound form of paclitaxel sold as ABRAXANE®) and doxetaxel (TAXOTERE®, Rhone-Poulenc Rorer, Antony, France); chlorambucil; gemcitabine; 6-thioguanine; mercaptopurine; methotrexate; folinic acid; platinum analogs such as cisplatin, oxaliplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitors, such as irinotecan and RFS 2000; difluoromethylomithine (DFMO); retinoic acid; and capecitabine; as well as their pharmaceutically acceptable salts.

Specific examples of chemotherapeutic agents include aclarubicin, actinomycin, alitretinon, altretamine, aminopterin, aminolevulinic acid, amrubicin, amsacrine, anagrelide, arsenic trioxide, asparaginase, atrasentan, belotecan, bexarotene, bendamustine, bleomycin, bortezomib, busulfan, camptothecin, capecitabine, carboplatin, carboquone, carmofur, carmustine, celecoxib, chlorambucil, chlormethine, cisplatin, cladribine, clofarabine, crisantaspase, cyclophosphamide, cytarabine, dacarbazine, dactinomycin, daunorubicin, decitabine, demecolcine, docetaxel, doxorubicin, efaproxiral, elesclomol, elsamitrucin, enocitabine, epirubicin, estramustine, etoglucid, etoposide, floxuridine, fludarabine, fluorouracil (5FU), fotemustine, gemcitabine, gliadel implants, hydroxycarbamide, hydroxyurea, idarubicin, ifosfamide, irinotecan, irofulven, ixabepilone, larotaxel, leucovorin, liposomal doxorubicin, liposomal daunorubicin, lonidamine, lomustine, lucanthone, mannosulfan, masoprocol, melphalan, mercaptopurine, mesna, methotrexate, methyl aminolevulinate, mitobronitol, mitoguazone, mitotane, mitomycin, mitoxantrone, nedaplatin, nimustine, oblimersen, omacetaxine, ortataxel, oxaliplatin, paclitaxel, pegaspargase, pemetrexed, pentostatin, pirarubicin, pixantrone, plicamycin, porfimer sodium, prednimustine, procarbazine, raltitrexed, ranimustine, rubitecan, sapacitabine, semustine, sitimagene ceradenovec, strataplatin, streptozocin, talaporfin, tegafur-uracil, temoporfin, temozolomide, teniposide, tesetaxel, testolactone, tetranitrate, thiotepa, tiazofurine, tioguanine, tipifarnib, topotecan, trabectedin, triaziquone, triethylenemelamine, triplatin, tretinoin, treosulfan, trofosfamide, uramustine, valrubicin, verteporfin, vinblastine, vincristine, vindesine, vinflunine, vinorelbine, vorinostat and zorubicin, or a pharmaceutically acceptable salt of the foregoing.

Numerous other therapies can also be administered during cancer treatment to mitigate the effects of the disease and/or side effects of the treatment, including therapies to manage pain (e.g., narcotics, acupuncture), gastric discomfort (e.g., antacids), dizziness (e.g., anti-vertigo medications), nausea (e.g., anti-nausea medications), infection (e.g., medications to increase red/white blood cell counts) and the like, all of which are readily appreciated by the person skilled in the art.

A compound of the disclosure or other therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. In some embodiments, administration (e.g., of a compound of the disclosure) is oral. In some embodiments, administration (e.g., of a compound of the disclosure) is intravenous. The preferred mode of administration can vary depending on the particular compound or agent. Typically, a compound of the disclosure or other therapeutic agent will be administered from about 1 to about 6 (e.g., 1, 2, 3, 4, 5 or 6) times per day, also or alternatively, as an infusion (e.g., a continuous infusion).

A compound of the disclosure or other therapeutic agent can be administered in a dosage ranging from about 0.001 mg/kg to about 100 mg/kg of body weight or, alternatively, in a dosage ranging from about 1 mg/dose to about 5,000 mg/dose, every 4 to 120 hours, or according to the requirements of the particular agent. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Alternatively, a compound of the disclosure or other therapeutic agent can be administered in a dosage ranging from about 10 mg/kg to about 200 mg/kg of body weight. For example, suitable dosages can be from about 20 mg/kg to about 200 mg/kg, from about 10 mg/kg to about 100 mg/kg, or from about 20 mg/kg to about 100 mg/kg body weight per treatment. Suitable dosages can be from about 1 mg/dose to about 5,000 mg/dose, from about 10 mg/dose to about 2,500 mg/dose or from about 100 mg/dose to about 1,000 mg/dose.

In some embodiments, suitable dosages of a compound of the disclosure (e.g., methanol, a methanol prodrug) produce plasma formate levels of less than 8 mM, e.g., less than 4 mM, less than 2 mM, less than 1 mM, less than 0.8 mM, less than 0.4 mM or less than 0.2 mM. In some embodiments, suitable dosages of methanol or a methanol prodrug provide less than 1.0 g/kg, e.g., less than 0.5 g/kg, less than 0.3 g/kg, less than 0.1 g/kg, less than 0.05 g/kg or less than 0.03 g/kg, methanol. In some embodiments, suitable dosages of methanol or a methanol prodrug produce plasma formate levels of less than 8 mM, e.g., less than 4 mM, less than 2 mM, less than 1 mM, less than 0.8 mM, less than 0.4 mM or less than 0.2 mM, and provide less than 1.0 g/kg, e.g., less than 0.5 g/kg, less than 0.3 g/kg, less than 0.1 g/kg, less than 0.05 g/kg or less than 0.03 g/kg, methanol.

Doses lower or higher than those recited above may be required. Specific dosage and treatment regimens for any particular patient will depend, for example, upon a variety of factors, such as the activity of the specific agent employed, the age, body weight, general health status, sex, diet, time of administration, rate of excretion, drug combination, the severity and course of the disease, condition or symptoms, the subject's disposition to the disease, condition or symptoms, and the judgment of the treating physician. Determining the dosage for a particular agent, subject and disease, disorder or condition is within the abilities of one of skill in the art.

Due to the potential toxicity of methanol and its metabolite, formate, it may be desirable to monitor a subject (e.g., a human) undergoing treatment with methanol, or a prodrug thereof, for signs and/or symptoms of methanol poisoning and/or formate toxicity. Thus, some embodiments further comprise monitoring a subject for methanol poisoning and/or formate toxicity (e.g., symptomatic toxicity) as, for example, by monitoring (e.g., quantifying) a formate level and/or pH level in a subject, for example, a plasma or serum formate level, or a blood gas pH level. Symptoms of methanol poisoning and/or formate toxicity include hyperventilation/dyspnea, visual disturbances, gastrointestinal symptoms, oxalate crystals in urine, chest pain and hangover-like symptoms. Other signs of methanol poisoning and/or formate toxicity include metabolic acidosis, increased anion gap, increased osmolar gap, and increased serum formate (e.g., as indicated by an arterial blood gas measurement). For example, a blood gas pH of 7.2 or less and/or formate level of 5 mM or greater may be used to indicate that treatment for methanol poisoning and/or formate toxicity should be administered.

Accordingly, some embodiments further comprise administering to a subject a treatment for methanol poisoning and/or formate toxicity as, for example, when methanol poisoning and/or formate toxicity is detected. Formate toxicity is detected in accordance with certain embodiments of the methods described herein when the subject has symptoms of toxicity, and/or when a blood gas pH of 7.2 or less and/or formate level of 5 mM or greater and/or methanol concentration of greater than 20 mg/dL.

Various treatments, such as antidotes, for methanol poisoning and/or formate toxicity are available, and include bicarbonate (e.g., sodium bicarbonate), ethanol, fomepizole, dialysis (e.g., intermittent hemodialysis (IHD), continuous veno-venous hemofiltration (CVVHD)), folinic acid, folic acid, ventilator support, and combinations of the foregoing. Bicarbonate, for example, can be given intravenously or orally (e.g., at a dose of from about 6 to about 10 tablets of 500 mg sodium bicarbonate every hour) until acidosis/hyperventilation is corrected. Ethanol can be given orally or intravenously (e.g., at a dose sufficient to achieve a serum ethanol concentration of from about 100 mg/dL to about 150 mg/dL; preferably continued for 12-24 hours after discontinuation of dialysis). Fomepizole can be given orally or intravenously (e.g., at an initial dose of 15 mg/kg followed by doses of 10 mg/kg every 12 hours or every 4 hours during hemodialysis; preferably continued for 12-24 hours after discontinuation of dialysis). IHD (e.g., intermittent, high-flow IHD) can be continued for at least about six hours (e.g., about 6 to about 8 hours). CVVHD can be continued for about 18 hours. Folinic acid can be given at a dose of 50 mg intravenously or orally every six hours for about 24 to about 48 hours. Ventilator support should be continued as long as a subject is acidotic.

In some embodiments, a treatment for methanol poisoning and/or formate toxicity comprises an inhibitor of ADH (e.g., fomepizole or ethanol). In some embodiments, a treatment for methanol poisoning and/or formate toxicity comprises an inhibitor of ADH (e.g., fomepizole or ethanol) and dialysis. In some embodiments, a treatment for methanol poisoning and/or formate toxicity comprises a buffer (e.g., bicarbonate, such as sodium bicarbonate) and an inhibitor of ADH (e.g., fomepizole or ethanol). In some embodiments, a treatment for methanol poisoning and/or formate toxicity comprises a buffer (e.g., bicarbonate, such as sodium bicarbonate), an inhibitor of ADH (e.g., fomepizole or ethanol) and dialysis. In some embodiments, a treatment for methanol poisoning and/or formate toxicity comprises a buffer (e.g., bicarbonate, such as sodium bicarbonate), an inhibitor of ADH (e.g., fomepizole or ethanol), dialysis and an agent that enhances metabolism of formate to carbon dioxide (e.g., folinic acid or folic acid). In some embodiments, a treatment for methanol poisoning and/or formate toxicity comprises one or more of a buffer (e.g., bicarbonate, such as sodium bicarbonate), an inhibitor of ADH (e.g., fomepizole or ethanol), dialysis and an agent that enhances metabolism of formate to carbon dioxide (e.g., folinic acid or folic acid).

Some embodiments comprise discontinuing (e.g., temporarily, permanently) administration of methanol, or a prodrug thereof, if methanol poisoning and/or formate toxicity is detected, e.g., in accordance with the methods described herein. Further embodiments comprise discontinuing administration of methanol, or a prodrug thereof, and administering a treatment for methanol poisoning and/or formate toxicity if methanol poisoning and/or formate toxicity is detected, e.g., in accordance with the methods described herein. In some embodiments, administration of methanol, or a prodrug thereof, may be resumed (e.g., at a lower dose) when methanol concentrations are undetectable or have been reduced below 20 mg/dL and/or the subject is asymptomatic with normal pH and/or formate level.

### EXEMPLIFICATION

### Example 1. MC38 Study

Female C57BL/6 mice (10 per group) were inoculated subcutaneously with 5×10⁵ MC38 syngeneic mouse tumor cells. Once the tumors had reached a volume of 80 to 120 mm³, the mice were randomized into treatment groups and dosing was initiated. All groups received anti-PD1 antibody (clone RMP1-14) via twice-weekly intraperitoneal injections at a dose of 5 mg per kg body weight for the first two weeks of dosing (e.g., four total doses), and one of the following treatments: vehicle (saline, dose volume of 10 mL per kg body weight) via daily oral gavage; methanol (HOCH₃, dissolved in saline to a concentration of 20% weight/volume, dose of 2 g methanol per kg body weight) via daily oral gavage; or sodium formate (dissolved in water to a concentration of 0.5% weight/volume) via supplementing the drinking water. Tumor volumes were measured by caliper twice weekly.

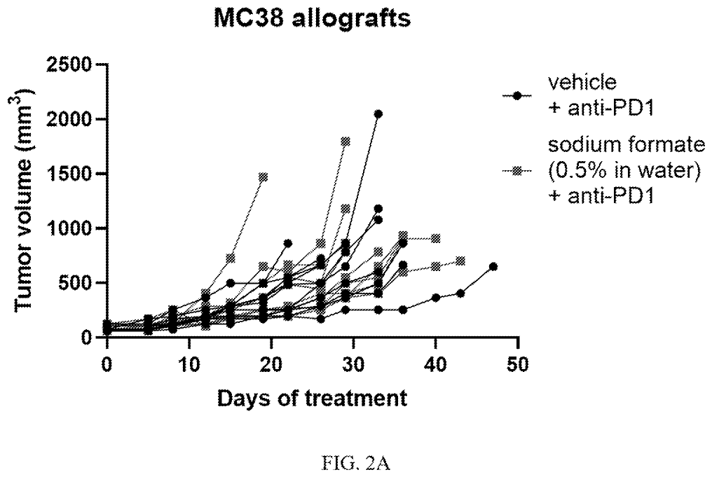

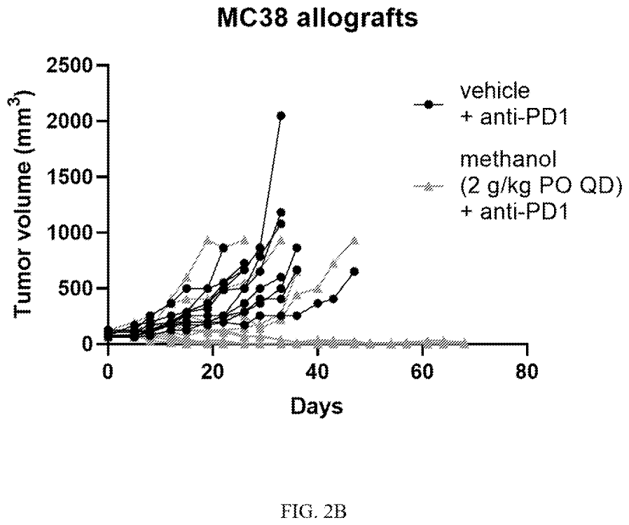

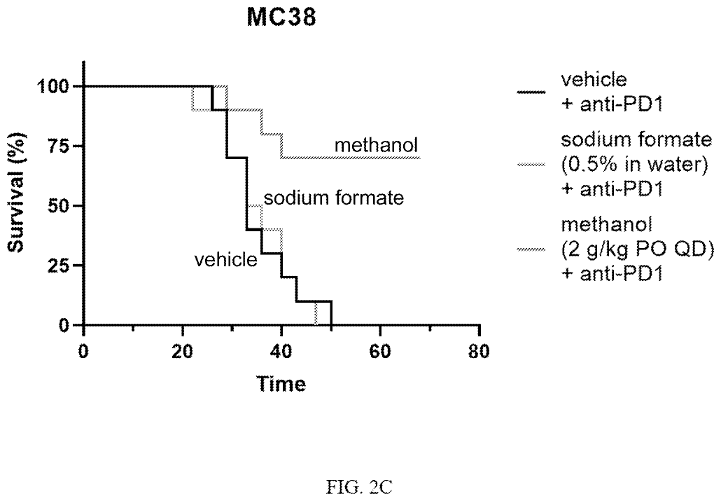

The results of the MC38 study are shown in FIGS. 2A-2C. An improved response to anti-PD1 therapy was observed in mice dosed with methanol compared to mice bearing syngeneic MC38 tumors that received vehicle or sodium formate. No effects on body weight were observed with either formate or methanol treatment.

### Example 2. MC38 Rechallenge

The 6 (out of 10 total) mice from Example 1 that demonstrated complete tumor clearance in response to the combination of methanol and anti-PD1 were rechallenged by inoculation with 5×10⁵ MC38 cells on the opposite flank. The rechallenged mice were monitored twice weekly for tumor growth for three weeks. Fifteen age-matched, tumor-naïve control mice were simultaneously inoculated as a control. The mice were not treated during the observation interval.

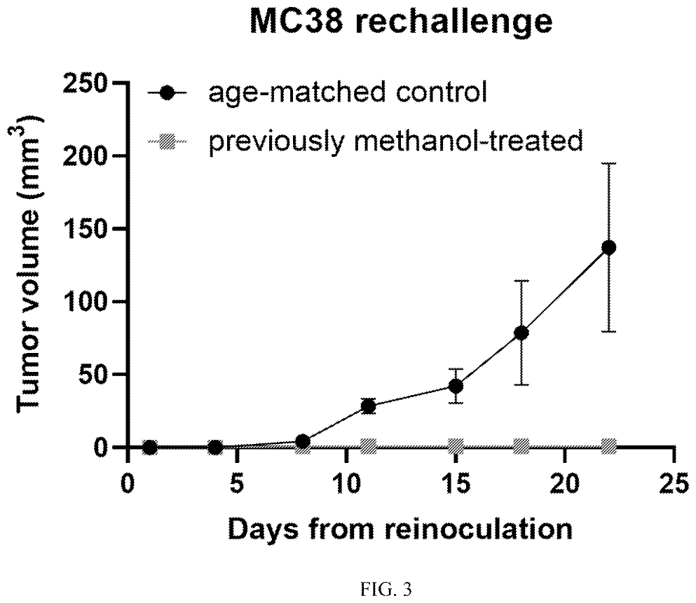

The results of the MC38 rechallenge are shown in FIG. 3. The rechallenged mice did not develop tumors during the observation interval, which demonstrates that the responses in the MC38 study described in Example 1 were immune-mediated, and the mice developed lasting anti-tumor immunity.

### Example 3. Synthetic Experimentals

Synthesis of methyl-d3 L-argininate dihydrochloride. To a solution of CD₃OD (1 mL) was added SOCl₂ (273 mg, 2.30 mmol, 2 equiv) dropwise at 0° C. The resulting mixture was stirred for 10 minutes at room temperature. To the resulting mixture was added L-arginine (200 mg, 1.15 mmol, 1 equiv). The resulting mixture was stirred for 16 hours at room temperature. The resulting mixture was concentrated under reduced pressure and the residue was dissolved in 20 mL distilled water. After lyophilization, methyl-d3 L-argininate dihydrochloride (90 mg, 34%) was obtained as a light-yellow solid.

LCMS Calculated for C₇H₁₅D₃Cl₂N₄O₂: 263.10, Observed: 191.1 [M+H−2HCl]⁺.

¹H-NMR: (400 MHz, DMSO-d₆, ppm): δ 8.64 (m, 3H), 7.90 (t, J=6.0 Hz, 1H), 7.27 (s, 4H), 4.05 (q, J=5.8 Hz, 1H), 3.14 (q, J=6.8 Hz, 2H), 1.93-1.74 (m, 2H), 1.57 (m, 2H).

### Synthesis of 1,1,1-tris(methoxy-d₃)butane

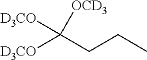

### Synthesis of methyl-d₃ butyrimidate hydrochloride

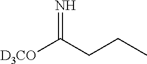

To a 250-mL, 3-necked, round-bottomed flask were added Et₂O (50 mL), butyronitrile (5 g, 72.352 mmol, 1 equiv) and CD₃OD (2.61 g, 72.352 mmol, 1 equiv) at room temperature. To this mixture was added HCl gas over 1 hour at 0° C. The resulting mixture was stirred for additional 1 hour at 0° C. The resulting mixture was concentrated under vacuum to afford methyl-d₃ butyrimidate hydrochloride (5 g, 49%) as a yellow solid. The crude product was used in the next step directly without further purification.

LCMS Calculated for C₅H₉D₃ClNO: 140.08, Observed: 104.1 [M+H]⁺.

Synthesis of 1,1,1-tris(methoxy-d₃)butane. To a 100-mL, round-bottomed flask were added CD₃OD (10 mL) and methyl butanimidate hydrochloride (5.00 g, 35.5 mmol, 1 equiv) at room temperature. The resulting mixture was stirred for 48 hours at room temperature under a nitrogen atmosphere. The resulting mixture was concentrated under reduced pressure. The crude product was purified by distillation under vacuum and the fraction was collected at 45° C. to afford 1,1,1-tris(methoxy-d₃)butane (90 mg, 1.6%) as a colorless liquid.

¹H-NMR: (400 MHz, DMSO-d₆, ppm): δ 2.31 (t, J=7.36 Hz, 2H), 1.69-1.60 (m, 2H), 0.96 (t, J=7.4 Hz, 3H).

### Example 4. Comparison of Undeuterated Methanol and Deuterated Methanol

Female C57BL/6 mice (13-15 per group) were inoculated subcutaneously with 5×10⁵ MC38 syngeneic mouse tumor cells. Once the tumors had reached a volume of 80 to 120 mm³, the mice were randomized into treatment groups and dosing was initiated. All groups received anti-PD1 antibody (clone RMP1-14, ichorbio, catalog number ICHi 132) via twice-weekly intraperitoneal injections at a dose of 5 mg per kg body weight for the first two weeks of dosing (e.g., four total doses), and one of the following treatments: vehicle (saline, dose volume of 10 mL per kg body weight) via daily oral gavage; methanol (HOCH₃, dissolved in saline to a concentration of 20% weight/volume, dose of 2 g HOCH₃ per kg body weight) via daily oral gavage; or methanol-d₃ (dissolved in saline to a concentration of 10% weight/volume, dose of 1 g methanol-d₃ per kg body weight) via daily oral gavage. Tumor volumes were measured by caliper twice weekly.

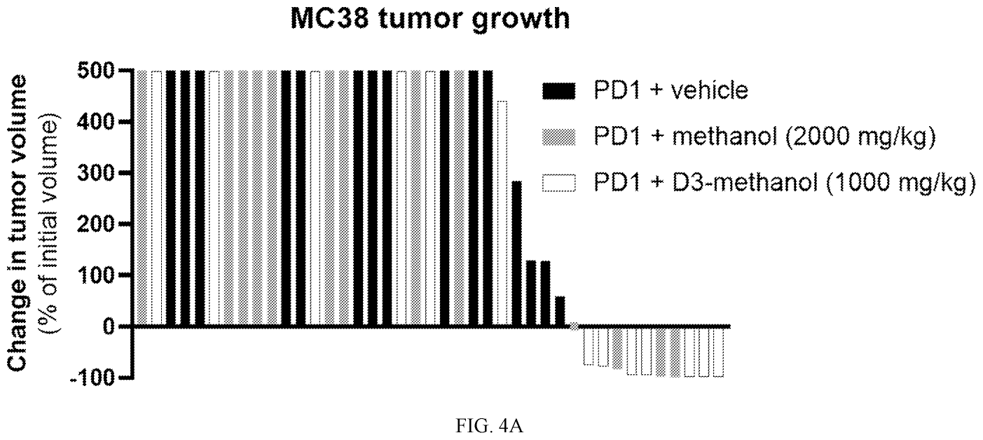

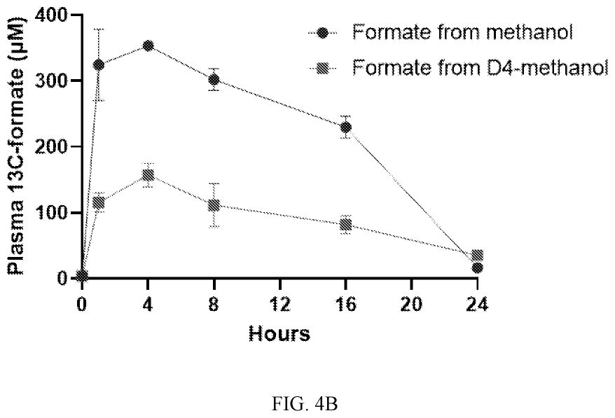

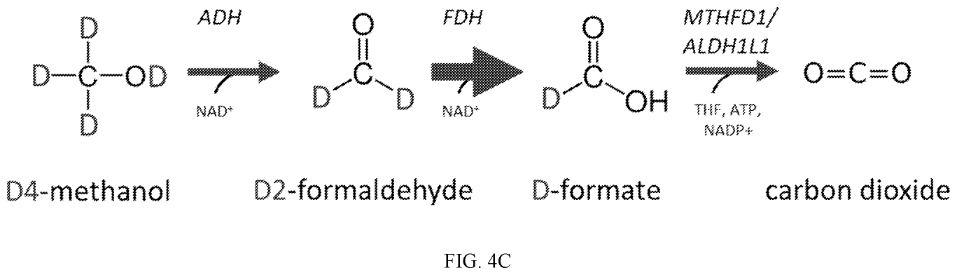

The results of the comparison of methanol and methanol-d₃ in the MC38 mouse model are shown in FIG. 4A.

In another experiment, female C57BL/6 mice (4 per group) were treated with a single dose of ¹³C-methanol (HO¹³CH₃, dissolved in saline to a concentration of 20% weight/volume, dose of 2 g HO¹³CH₃ per kg body weight) via oral gavage, or ¹³C-methanol-d₄ (dissolved in saline to a concentration of 20% weight/volume, dose of 2 g ¹³C-methanol-d₄ per kg body weight) via oral gavage. Serum samples were collected from the retro-orbital vein at 1, 4, 8, and 16 hours post-dose, and terminal samples were collected by cardiac puncture at 24 hours post-dose. Formate levels in the serum samples were determined by tandem liquid chromatography-mass spectrometry (LC-MS) analysis.

The results of the comparison of ¹³C-methanol and ¹³C-methanol-d₄ by LC-MS in serum are plotted in FIG. 4B.

Without wishing to be bound by any particular theory, it is believed that kinetic isotope effects of deuterium slow rates of hydrogen abstraction in liver metabolism, thereby slowing both formate production and clearance. FIG. 4C depicts expected liver metabolism of methanol-d₄. Taken together, FIGS. 4A-4C suggest that the rate of formate production and clearance can be controlled with deuteration of methanol.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating cancer in a subject in need thereof, comprising administering to the subject a therapeutically effective amount of methanol, or a prodrug thereof.

2. The method of claim 1, further comprising administering to the subject an additional therapeutic agent.

3. The method of claim 2, wherein the additional therapeutic agent is an immunotherapy.

4. A method for enhancing an immunotherapy in a subject in need thereof, comprising administering to the subject a therapeutically effective amount of methanol, or a prodrug thereof.

5. The method of claim 4, further comprising administering to the subject the immunotherapy.

6. The method of claim 3, 4 or 5, wherein the immunotherapy comprises a checkpoint inhibitor.

7. The method of claim 6, wherein the checkpoint inhibitor is an inhibitor of CTLA-4, PD-1, PD-L1 or LAG-3.

8. The method of claim 7, wherein the checkpoint inhibitor is a PD-1 inhibitor.

9. The method of claim 8, wherein the PD-1 inhibitor is nivolumab, pembrolizumab or cemiplimab.

10. The method of any one of claims 7-9, wherein the checkpoint inhibitor is a PD-L1 inhibitor.

11. The method of claim 10, wherein the PD-L1 inhibitor is avelumab, durvalumab or atezolizumab.

12. The method of any one of claims 4-11, wherein the subject has cancer.

13. The method of any one of claims 1-3 and 12, wherein the cancer is a solid tumor cancer.

14. The method of any one of claims 1-3 and 12, wherein the cancer is a hematologic cancer.

15. The method of any one of claims 1-3 and 12, wherein the cancer is breast cancer, bladder cancer, cervical cancer, colon cancer, head and neck cancer, lymphoma, liver cancer, lung cancer, kidney cancer, skin cancer, stomach cancer, esophageal cancer, rectal cancer, pancreatic cancer, ovarian cancer or a solid tumor that is not able to repair errors in its DNA that occur when the DNA is copied.

16. The method of any one of claims 1-15, comprising administering to the subject a therapeutically effective amount of methanol.

17. The method of claim 16, wherein the methanol is deuterated.

18. The method of claim 16, wherein the methanol is HOCH₃, HOCD₃, DOCD₃, HO¹³CH₃, HO¹³CD₃ or DO¹³CD₃.

19. The method of any one of claims 1-15, comprising administering to the subject a therapeutically effective amount of a prodrug of methanol.

20. The method of any one of claims 1-19, wherein the methanol or prodrug of methanol is administered orally.

21. The method of any one of claims 1-20, further comprising monitoring the subject for methanol poisoning or formate toxicity.

22. The method of any one of claims 1-21, further comprising administering to the subject a treatment for methanol poisoning or formate toxicity if methanol poisoning or formate toxicity is detected.

23. The method of any one of claims 1-22, further comprising discontinuing administration of methanol or a prodrug of methanol if methanol poisoning or formate toxicity is detected.

24. A compound of the following structural formula: 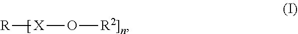
or a pharmaceutically acceptable salt thereof, wherein:
each X is independently —C(O)—, —Y—C(O)—, —C(O)—Y—C(O)—, —Y—CH₂—, —C(O)—Y—CH₂— or a covalent bond;
each Y is independently O or N(R¹);
each R¹ is independently —H, formyl, —CO₂H, —CO₂R², —CH₂—OR², —CO₂(C₂-C₅)alkyl or —CO₂CH₂—(C₆-C₁₅)aryl; or
R is —H, -D, —O*CH₃, —O*CD₃, —O*CH₂D or —O*CHD₂, or (C₁-C₁₅)alkyl or (C₁-C₁₅)alkylether substituted with -(R¹⁰)ₘ;
each R¹⁰ is independently halo, hydroxy, (C₁-C₅)alkoxy, —CO₂H, —CO₂(C₂-C₅)alkyl, —O-formyl or —NR¹¹R¹²
m is 0, 1, 2, 3, 4 or 5;
R¹¹ and R¹² are each independently —H, formyl, —CO₂H, —CO₂(C₂-C₅)alkyl or —CO₂CH₂—(C₆-C₁₅)aryl;
each R² is independently —*CH₃, —*CD₃, —*CH₂D or —*CHD₂;
n is an integer from 1 to 25, inclusive;
each carbon atom indicated with an * is independently ¹²C or 13C;
each carbon atom of a hydrolyzable formyl is independently ¹²C or ¹³C; and
each hydrogen atom of a hydrolyzable formyl is H or D,
provided:
(i) when X is a covalent bond, there are at least two occurrences of X attached to a single carbon atom of R which are a covalent bond,
(ii) if n is 1, then R is —H, -D, —O*CH₃, —O*CD₃, —O*CH₂D or —O*CHD₂, or the compound contains at least one hydrolyzable formyl,
(iii) the compound is not dimethyladipate, trimethylorthoformate, trimethylorthoacetate, tetramethoxymethane, trimethoxymethane-d, trimethoxymethane-¹³C, (bis(methoxy-d₃)methoxy-d)methane-d₃, 1,1,1,2,2-pentamethoxypropane, 1,1,1,3,3,3-hexamethoxypropane, 1,1,1,4,4,4-hexamethoxybutane, 2,2,2-trimethoxyethan-1-amine, 1,1,1-trimethoxypropan-2-amine, 1,1,1-trichloro-2,2,2-trimethoxyethane or 1,1,1-trifluoro-2,2,2-trimethoxyethane, or a salt of the foregoing, and
(iv) R is not —H or -D when X is —C(O)— and n is 1.

25. The compound of claim 24, wherein each Y is O.

26. The compound of claim 24, wherein each Y is N(R¹).

27. The compound of any one of claims 24-26, wherein each R¹ is independently formyl or —CO₂R².

28. The compound of any one of claims 24-27, wherein R is (C₁-C₁₅)alkyl substituted with (R¹⁰)ₘ.

29. The compound of claim 28, wherein R is (C₁-C₁₀)alkyl substituted with (R¹⁰ₘ.

30. The compound of claim 29, wherein R is (C₁-C₅)alkyl substituted with (R¹⁰ₘ.

31. The compound of any one of claims 24-27, wherein R is (C₁-C₁₅)alkylether substituted with (R¹⁰)ₘ.

32. The compound of any one of claims 24-27, wherein R is —H or -D.

33. The compound of any one of claims 24-27, wherein R is —O*CH₃ or —O*CD₃.

34. The compound of any one of claims 24-31, wherein each R¹⁰ is independently —CO₂H, —O-formyl or —NR¹¹R¹².

35. The compound of any one of claims 24-31 and 34, wherein R¹¹ and R¹² are each independently H, formyl or —CO₂(C₂-C₅)alkyl.

36. The compound of any one of claims 24-35, wherein n is an integer from 1 to 8, inclusive.

37. The compound of any one of claims 24-35, wherein n is an integer from 2 to 10, inclusive.

38. The compound of any one of claims 24-35, wherein n is an integer from 1 to 5, inclusive.

39. A compound of Structural Formula II: 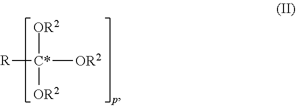
or a pharmaceutically acceptable salt thereof, wherein:
R is H or D, or (C₁-C₁₅)alkyl substituted with -(R²⁰)ₘ;
each R²⁰ is independently halo, hydroxy, —OR², (C₂-C₅)alkoxy, —CO₂H, —CO₂R², —CO₂(C₂-C₅)alkyl, —O-formyl or —NR²¹R²²;
m is 0, 1, 2, 3, 4 or 5;
R²¹ and R²² are each independently —H, formyl, —CO₂H, —CO₂R², —CO₂(C₂-C₅)alkyl or —CO₂CH₂—(C₆-C₁₅)aryl;
each R² is independently —*CH₃, —*CD₃, —*CH₂D or —*CHD₂;
p is an integer from 1 to 10, inclusive;
each carbon atom indicated with an * is independently ¹²C or 13C;
each carbon atom of a hydrolyzable formyl is independently ¹²C or ¹³C; and
each hydrogen atom of a hydrolyzable formyl is H or D,
provided the compound is not trimethylorthoformate, trimethoxymethane-d, trimethoxymethane-¹³C, (bis(methoxy-d₃)methoxy-d)methane-d₃, trimethylorthoacetate, 1,1,1,2,2-pentamethoxypropane, 1,1,1,3,3,3-hexamethoxypropane, 1,1,1,4,4,4-hexamethoxybutane, 2,2,2-trimethoxyethan-1-amine, 1,1,1-trimethoxypropan-2-amine, 1,1,1-trichloro-2,2,2-trimethoxyethane or 1,1,1-trifluoro-2,2,2-trimethoxyethane, or a salt of any of the foregoing.

40. The compound of claim 39, wherein R is H or D.

41. The compound of claim 39, wherein R is (C₁-C₁₅)alkyl substituted with -(R²⁰)ₘ.

42. The compound of claim 41, wherein R is (C₁-C₁₀)alkyl substituted with -(R²⁰)ₘ.

43. The compound of claim 42, wherein R is (C₁-C₅)alkyl substituted with -(R²⁰)ₘ.

44. The compound of any one of claims 39 and 41-43, wherein each R²⁰ is independently halo, hydroxy, —O—R² or —NR²¹R²².

45. The compound of claim 44, wherein each R²⁰ is independently fluoro, chloro, hydroxy, —O—R² or —NH₂.

46. The compound of any one of claims 39 and 41-44, wherein R²¹ and R²² are each independently H, formyl or —CO₂R².

47. The compound of any one of claims 24-31, 34-39 and 41-46, wherein m is 0, 1, 2 or 3.

48. The compound of claim 47, wherein m is 0.

49. The compound of any one of claims 24-31, 34-39 and 41-48, wherein R is a residue of a metabolite, an amino acid, a polyol or a food additive.

50. The compound of claim 49, wherein R is a metabolite residue.

51. The compound of claim 50, wherein the metabolite is citric acid, isocitric acid, aconitic acid, alpha-ketoglutaric acid, fumaric acid, malic acid, succinic acid, lactic acid or pyruvic acid.

52. The compound of claim 49, wherein R is an amino acid residue.

53. The compound of claim 52, wherein the amino acid is glycine, alanine, lysine, aspartic acid, glutamic acid, serine, threonine, asparagine or glutamine.

54. The compound of claim 49, wherein R is a polyol residue.

55. The compound of claim 54, wherein the polyol is glycerol, erythritol, xylitol, sorbitol, ribose, 2-deoxyribose, fructose, glucose, galactose, mannose, allose, altrose, gulose, idose, talose, xylose, maltitol, isomalt or sucrose.

56. The compound of claim 49, wherein R is a food additive residue.

57. The compound of claim 56, wherein the food additive is tartaric acid.

58. The compound of any one of claims 24-31, 34-39 and 41-48, wherein R is a residue of an endogenous carboxylic acid.

59. The compound of any one of claims 24-31, 34-39 and 41-48, wherein R is a residue of formic acid, pyruvic acid, oxoglutaric acid (e.g., 2-oxoglutaric acid), oxalosuccinic acid, malonic acid, citric acid, 1-hydroxypropane-1,2,3-tricarboxylic acid, succinic acid, 2-oxosuccinic acid, glycine, glutamic acid, aspartic acid, alanine, trichloroacetic acid or trifluoroacetic acid.

60. The compound of any one of claims 39-59, wherein p is an integer from 1 to 5, inclusive.

61. The compound of any one of claims 24-60, wherein each R² is independently —*CH₃ or —*CD₃.

62. The compound of claim 61, wherein each R² is —*CH₃.

63. The compound of claim 61, wherein each R² is —*CD₃.

64. The compound of any one of claims 24-63, wherein each carbon atom indicated with an asterisk is ¹²C.

65. The compound of any one of claims 24-63, wherein each carbon atom indicated with an asterisk is ¹³C.

66. The compound of any one of claims 24-65, wherein each carbon atom of a hydrolyzable formyl is ¹²C.

67. The compound of any one of claims 24-65, wherein each carbon atom of a hydrolyzable formyl is ¹³C.

68. The compound of any one of claims 24-67, wherein each hydrogen atom of a hydrolyzable formyl is H.

69. The compound of any one of claims 24-67, wherein each hydrogen atom of a hydrolyzable formyl is D.

70. A pharmaceutical composition comprising a compound of any one of claims 24-69, or a pharmaceutically acceptable salt thereof, and a pharmaceutically acceptable carrier.

71. A pharmaceutical composition comprising (i) methanol, or a prodrug thereof, or (ii) a compound of any one of claims 24-69, or a pharmaceutically acceptable salt thereof, and an additional therapeutic agent.

72. A pharmaceutical combination comprising (i) methanol, or a prodrug thereof, or (ii) a compound of any one of claims 24-69, or a pharmaceutically acceptable salt thereof, and an additional therapeutic agent.

73. The pharmaceutical composition of claim 71 or pharmaceutical combination of claim 72, wherein the additional therapeutic agent is a checkpoint inhibitor.

74. The pharmaceutical composition or pharmaceutical combination of claim 73, wherein the checkpoint inhibitor is an inhibitor of CTLA-4, PD-1, PD-L1 or LAG-3.

75. The pharmaceutical composition or pharmaceutical combination of claim 74, wherein the checkpoint inhibitor is a PD-1 inhibitor.

76. The pharmaceutical composition or pharmaceutical combination of claim 75, wherein the PD-1 inhibitor is nivolumab, pembrolizumab or cemiplimab.

77. The pharmaceutical composition or pharmaceutical combination of any one of claims 73-76, wherein the checkpoint inhibitor is a PD-L1 inhibitor.

78. The pharmaceutical composition or pharmaceutical combination of claim 77, wherein the PD-L1 inhibitor is avelumab, durvalumab or atezolizumab.

79. A method of treating cancer in a subject in need thereof, comprising administering to the subject a therapeutically effective amount of a compound of any one of claims 24-69, or a pharmaceutically acceptable salt thereof, a pharmaceutical composition of any one of claims 70, 71 and 73-78; or a pharmaceutical combination of any one of claims 72-78.

80. A method for enhancing an immunotherapy in a subject in need thereof, comprising administering to the subject a therapeutically effective amount of a compound of any one of claims 24-69, or a pharmaceutically acceptable salt thereof, a pharmaceutical composition of any one of claims 70, 71 and 73-78; or a pharmaceutical combination of any one of claims 72-78.
